(19) United States
(12) Patent Application Publication
(10) Pub. No.: US 2018/0111021 A1
(43) Pub. Date: Apr. 26, 2018

(54) EXERCISE ANALYSIS DEVICE, EXERCISE ANALYSIS SYSTEM, AND EXERCISE ANALYSIS METHOD

(71) Applicant: SEIKO EPSON CORPORATION, Tokyo (JP)

(72) Inventors: Kazumi MATSUMOTO, Shiojiri-shi (JP); Shunichi MIZUOCHI, Matsumoto-shi (JP); Shuji UCHIDA, Shiojiri-shi (JP)

(73) Assignee: SEIKO EPSON CORPORATION, Tokyo (JP)

(21) Appl. No.: 15/729,134

(22) Filed: Oct. 10, 2017

(30) Foreign Application Priority Data
Oct. 25, 2016 (JP) 2016-208385

(51) Int. Cl. A63B 24/00; G01C 21/16; A61B 5/107; A61B 5/11; G01P 15/18; G01C 22/00; G06K 9/00

(52) U.S. Cl. CPC A63B 24/0006; G01C 21/16; A61B 5/107; A61B 2562/0219; G01P 15/18; G01C 22/006; G06K 9/00348; A61B 5/1122

(57) ABSTRACT

An exercise analysis device includes: an exercise analysis unit that generates exercise information during running or walking of a subject using output of an inertial measurement unit (IMU); and an output unit that converts exercise information periodically generated among the exercise information into predetermined perceptual information and outputs the perceptual information in synchronization with landing.

### BACKGROUND

### 1. Technical Field

The present invention relates to an exercise analysis device, an exercise analysis system, and an exercise analysis method.

### 2. Related Art

In general, a device that measures and presents various indexes during exercise is known. JP-T-2013-537436 discloses a device that calculates and displays bio-mechanical parameters of a stride of a runner based on acceleration data. As the bio-mechanical parameters, a landing angle of a leg onto a ground, a moving distance of a gravity center of the runner during a foot contact with the ground, and the like are described.

However, the device described in JP-T-2013-537436 integrally includes a calculation unit that calculates bio-mechanical parameters and a display unit that displays the calculated bio-mechanical parameters, and is mounted on the runner's waist to accurately detect acceleration data. For this reason, it was difficult for the runner during running to keep checking displayed indexes and to run while understanding the presented indexes.

### SUMMARY

An advantage of some aspects of the invention is to solve at least a part of the problems described above, and the invention can be implemented as the following forms or application examples.

### APPLICATION EXAMPLE 1

An exercise analysis device according to this application example includes: an exercise information generation unit that generates exercise information during running or walking of a subject using output of an inertial sensor; and an output unit that converts exercise information periodically generated among the exercise information into predetermined perceptual information and outputs the perceptual information in synchronization with landing.

According to this application example, it is possible to convert the exercise information periodically generated base on output of the inertial sensor and to output the perceptual information in synchronization with landing. For this reason, even when the subject is exercising such as running or walking, since the exercise information of the subject is converted and reported as perceptual information, it is possible to easily check the exercise information during exercise of the subject. Accordingly, it is possible for the subject to modify an exercise state and the like during exercise according to the exercise information.

### APPLICATION EXAMPLE 2

In the exercise analysis device according to the application example, it is preferable that the perceptual information is at least one of sound, light, or vibration.

According to this application example, since the perceptual information is at least one of sound, light, or vibration, it is possible to check the exercise information sensately without relying on vision.

### APPLICATION EXAMPLE 3

In the exercise analysis device according to the application example, it is preferable that the exercise information includes information related to speed or acceleration in exercise of the subject.

According to this application example, since the exercise information includes information related to speed or acceleration during running or walking of the subject, it is possible to check the exercise information related to the speed or the acceleration of the subject.

### APPLICATION EXAMPLE 4

In the exercise analysis device according to the application example, it is preferable that the exercise information is converted into sound, light, or vibration having a frequency corresponding to the speed or the acceleration and is output.

According to this application example, the exercise information is converted into sound, light, or vibration having a frequency corresponding to the speed or the acceleration and is output, and thus it is possible to check a difference or magnitude of the speed or the acceleration as a difference or magnitude of the frequency.

### APPLICATION EXAMPLE 5

In the exercise analysis device according to the application example, it is preferable that the exercise information includes information related to a stride, a pitch, propulsion efficiency, an amount of brake at the time of landing, or ground time during running of the subject.

According to this application example, since the exercise information includes information related to a stride, a pitch, propulsion efficiency, an amount of brake at the time of landing, or ground time during running or walking of the subject, it is possible to check the exercise information of the subject in detail.

### APPLICATION EXAMPLE 6

In the exercise analysis device according to the application example, it is preferable that the perceptual information is output at the time of landing.

According to this application example, by outputting the perceptual information at the time of landing, it is possible to check the exercise information while maintaining rhythm of the subject during running or walking.

### APPLICATION EXAMPLE 7

In the exercise analysis device according to the application example, it is preferable that the perceptual information is output within ±100 ms at the time of landing.

According to this application example, by outputting the perceptual information within ±100 ms at the time of landing, it is possible to check the exercise information while more accurately maintaining rhythm of the subject during running or walking.

### APPLICATION EXAMPLE 8

In the exercise analysis device according to the application example, it is preferable that the sound includes mimetic sound.

According to this application example, since the sound of the perceptual information includes mimetic sound and the sound as the perceptual information becomes easier to hear, it is possible to more accurately check the exercise information.

### APPLICATION EXAMPLE 9

In the exercise analysis device according to the application example, it is preferable that the exercise information is output as different perceptual information on a left foot and a right foot of the subject.

According to this application example, the exercise information is output as different perceptual information on the left foot and the right foot of the subject, and thus it is possible for the subject to easily determine and check whether the exercise information is information on the left foot or the right foot.

### APPLICATION EXAMPLE 10

An exercise analysis system according to this application example includes: an exercise analysis device that includes an exercise information generation unit that generates exercise information during running or walking of a subject using output of an inertial sensor, and an output unit that converts exercise information periodically generated among the exercise information into predetermined perceptual information and outputs the perceptual information in synchronization with landing; and a reporting device that reports the exercise information.

According to this application example, since the exercise analysis device includes the output unit that converts the exercise information periodically generated based on output of the inertial sensor into perceptual information and outputs the perceptual information in synchronization with landing, by outputting the exercise information as the perceptual information, even when the subject is exercising such as running or walking, the exercise information of the subject is converted and reported as perceptual information, and thus it is possible to easily check the exercise information during exercise of the subject. For this reason, it is possible for the subject, for example, to modify an exercise state during exercise according to the exercise information.

### APPLICATION EXAMPLE 11

An exercise analysis method according to this application example includes: generating exercise information during running or walking of a subject using output of an inertial sensor; and converting exercise information periodically generated among the exercise information into predetermined perceptual information and outputting the perceptual information in synchronization with landing.

According to this application example, since the exercise analysis method includes converting the exercise information periodically generated based on output of the inertial sensor into perceptual information and outputtting the perceptual information in synchronization with landing, by outputting the exercise information as the perceptual information, even when the subject is exercising such as running or walking, the exercise information of the subject is converted and reported as perceptual information, and thus it is possible to easily check the exercise information during exercise of the subject. For this reason, it is possible, for example, to modify an exercise state during exercise according to the exercise information.

### BRIEF DESCRIPTION OF THE DRAWINGS

The invention will be described with reference to the accompanying drawings, wherein like numbers reference like elements.

FIG. 1 is an illustrative diagram of an overview of an exercise analysis system according to an embodiment.

FIG. 2 is a functional block diagram illustrating an example of a configuration of an exercise analysis device according to the embodiment.

FIG. 3 is a diagram illustrating an example of a configuration of a sensing data table.

FIG. 4 is a diagram illustrating an example of a configuration of a GPS data table.

FIG. 5 is a diagram illustrating an example of a configuration of a geomagnetic data table.

FIG. 6 is a diagram illustrating an example of a configuration of a calculation data table.

FIG. 7 is a functional block diagram illustrating an example of a configuration of a processing unit of the exercise analysis device of the embodiment.

FIG. 8 is a functional block diagram illustrating an example of a configuration of an inertial navigation operation unit.

FIG. 9 is an illustrative diagram of a posture at the time of running of a user.

FIG. 10 is an illustrative diagram of a yaw angle at the time of running of the user.

FIG. 11 is a diagram illustrating an example of 3-axis acceleration at the time of running of the user.

FIG. 12 is a functional block diagram illustrating an example of a configuration of the exercise analysis device according to the embodiment.

FIG. 13 is a flowchart diagram illustrating an example of a procedure of an exercise analysis process according to the embodiment.

FIG. 14 is a flowchart diagram illustrating an example of a procedure of an inertial navigation operation process.

FIG. 15 is a flowchart diagram illustrating an example of a procedure of a running detection process.

FIG. 16 is a flowchart diagram illustrating an example of a procedure of an exercise analysis information generation process.

### DESCRIPTION OF EXEMPLARY EMBODIMENTS

Hereinafter, preferred embodiments of the invention will be described in detail with reference to the accompanying drawings. Also, the embodiments described hereinafter do not unfairly limit the content of the invention described in the appended claims. Further, not all of configurations described hereinafter are essential configuration requirements of the invention.

### Embodiment

### 1. Overview of Exercise Analysis System

Hereinafter, an exercise analysis system that analyzes exercise in running (including walking) of a user as a subject will be described byway of example, but an exercise analysis system of the present embodiment can apply to an exercise analysis system that analyzes exercise other than running in the same manner.

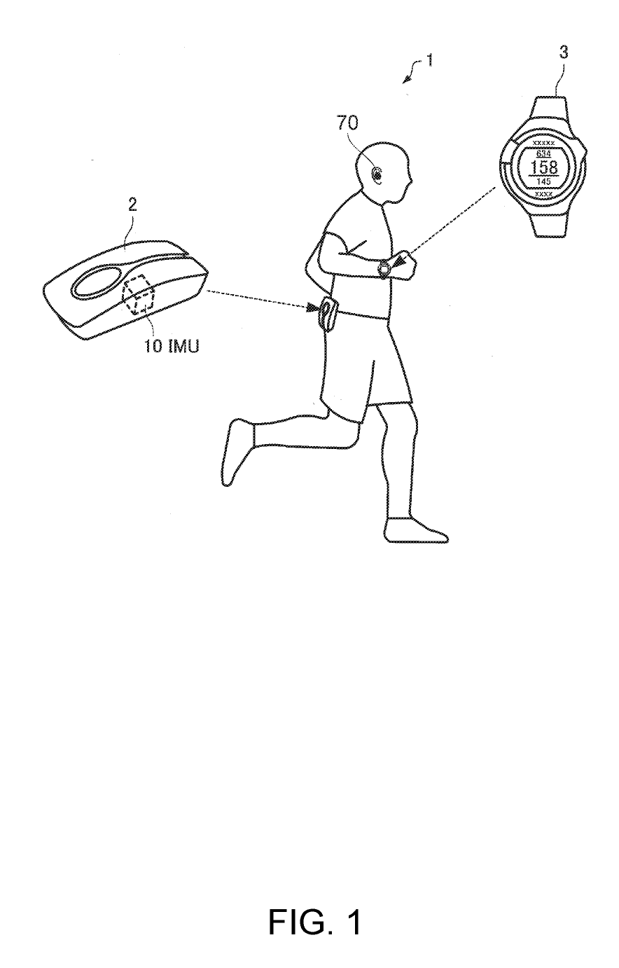

FIG. 1 is a diagram illustrating an example of a configuration of an exercise analysis system 1 of the present embodiment.

As illustrated in FIG. 1, the exercise analysis system 1 of the present embodiment is configured to include an exercise analysis device 2 and a reporting device 3. The exercise analysis device 2 is a device that analyzes exercise during running of the user, converts exercise information into predetermined perceptual information, and outputs the perceptual information. In addition, the reporting device 3 is a device that notifies the user of information on an exercise state during running of the user or a running result. In the present embodiment, as illustrated in FIG. 1, the exercise analysis device 2 includes an inertial measurement unit (IMU) 10 and is mounted to a torso portion (for example, a right waist, a left waist, or a central portion of a waist) of the user so that one detection axis (hereinafter, referred to as “z-axis”) of the inertial measurement unit (IMU) 10 substantially matches a gravitational acceleration direction (vertically downward) in a state in which the user is at rest, and an output unit 70 such as an earphone is mounted on a head (for example, an ear) of the user. In addition, the reporting device 3 is a wrist type (wristwatch type) portable information device, and is mounted on, for example, the wrist of the user. However, the reporting device 3 may be a portable information device, such as a head mount display (HMD) or a smartphone.

The user operates the reporting device 3 at the time of running start to instruct the exercise analysis device 2 to start measurement (inertial navigation operation process and exercise analysis process to be described below), and operates the reporting device 3 at the time of running end to instruct to end the measurement in the exercise analysis device 2. The reporting device 3 transmits a command for instructing start or end of the measurement to the exercise analysis device 2 in response to the operation of the user. In addition, the reporting device 3 selects exercise information to be output as perceptual information such as sound or vibration, and transmits the exercise information to the exercise analysis device 2.

When the exercise analysis device 2 receives the measurement start command, the exercise analysis device 2 starts the measurement using an inertial measurement unit (IMU) 10, analyzes a running state of the user using a measurement result, calculates values for various exercise indexes which are indexes regarding running capability (an example of exercise capability) of the user, and generates exercise information (hereinafter, referred to as “exercise analysis information”) including the values of the various exercise indexes as information on the analysis result of the running exercise of the user. The exercise analysis device 2 generates information to be output during running of the user (output information during running) using the generated exercise analysis information, and transmits the information to the output unit 70 and the reporting device 3. The output unit 70 converts the selected exercise analysis information into perceptual information such as sound or vibration and outputs the perceptual information, and reports goodness or badness of the exercise indexes to the user by the perceptual information. For this reason, even when the user is running, it is possible to easily check the exercise analysis information. The reporting device 3 receives the output information during running from the exercise analysis device 2, compares values of various exercise indexes included in the output information during running with preset target values, and reports the comparison result to the user, so that the user can run while checking goodness or badness of each of the exercise indexes.

Further, when the exercise analysis device 2 receives the measurement end command, the exercise analysis device 2 ends the measurement of the inertial measurement unit (IMU) 10, generates user running result information (running result information: running distance and running speed), and transmits the user running result information to the reporting device 3. The reporting device 3 receives the running result information from the exercise analysis device 2, and reports running result information to the user as a text or an image. Accordingly, the user can recognize the running result information immediately after the running end. Alternatively, the reporting device 3 may generate running result information based on the output information during running and may report the running result information to the user as a text or an image.

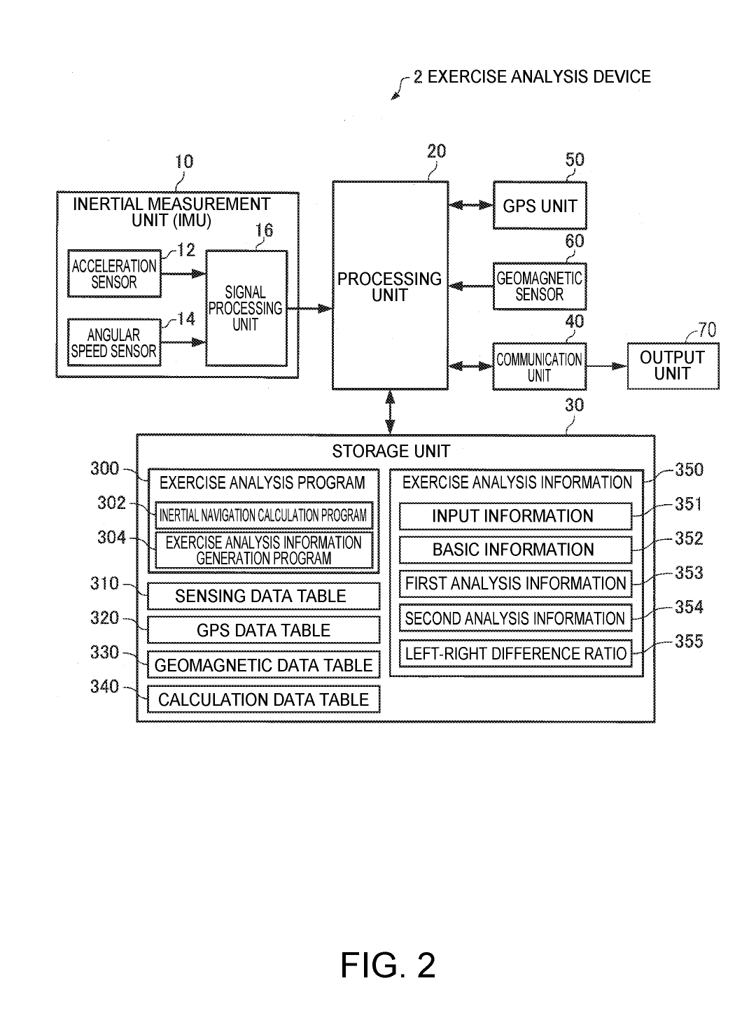

Also, data communication between a communication unit 40 (see FIG. 2) of the exercise analysis device 2 and the output unit 70 or the reporting device 3 may be wireless communication or may be wired communication.

### 2. Coordinate System

Coordinate systems required in the following description will be defined.

Earth Centered Earth Fixed Frame (e frame): A right-handed, three-dimensional orthogonal coordinate system in which a center of the Earth is an origin, and a z axis is parallel to a rotation axis.

Navigation Frame (n frame): A three-dimensional orthogonal coordinate system in which a moving object (user) is an origin, an x axis is north, a y axis is east, and a z axis is a gravity direction.

Body Frame (b frame): A three-dimensional orthogonal coordinate system in which a sensor (inertial measurement unit (IMU) 10) is a reference.

Moving Frame (m frame): A right-handed, three-dimensional orthogonal coordinate system in which a moving object (user) is an origin, and a running direction of the moving object (user) is an x axis.

### 3. Exercise Analysis Device

### 3-1. Configuration of Exercise Analysis Device

FIG. 2 is a functional block diagram illustrating an example of a configuration of the exercise analysis device 2 according to the present embodiment. As illustrated in FIG. 2, the exercise analysis device 2 is configured to include the inertial measurement unit (IMU) 10, a processing unit 20, a storage unit 30, the communication unit 40, a global positioning system (GPS) unit 50, a geomagnetic sensor 60, and the output unit 70. However, in the exercise analysis device 2 of the present embodiment, some of these components may be removed or changed, or other components may be added.

The inertial measurement unit 10 (an example of an inertial sensor) is configured to include an acceleration sensor 12, an angular speed sensor 14, and a signal processing unit 16.

The acceleration sensor 12 detects respective accelerations in 3-axis directions crossing one another (ideally, orthogonal to one another), and outputs a digital signal (acceleration data) according to magnitudes and directions of the detected 3-axis accelerations.

The angular speed sensor 14 detects respective angular speeds in 3-axis directions crossing one another (ideally, orthogonal to one another), and outputs a digital signal (angular speed data) according to magnitudes and directions of the measured 3-axis angular speed.

The signal processing unit 16 receives the acceleration data and the angular speed data from the acceleration sensor 12 and the angular speed sensor 14, attaches time information to the acceleration data and the angular speed data, stores the acceleration data and the angular speed data in a storage unit (not illustrated), generates sensing data obtained by causing the stored acceleration data, angular speed data, and time information to conform to a predetermined format, and outputs the sensing data to the processing unit 20.

The acceleration sensor 12 and the angular speed sensor 14 are ideally attached so that the three axes match three axes of the sensor coordinate system (b frame) relative to the inertial measurement unit 10, but an error of an attachment angle is actually generated. Therefore, the signal processing unit 16 performs a process of converting the acceleration data and the angular speed data into data of the sensor coordinate system (b frame) using a correction parameter calculated according to the attachment angle error in advance. Also, the processing unit 20 to be described below may perform the conversion process in place of the signal processing unit 16.

Further, the signal processing unit 16 may perform a temperature correction process for the acceleration sensor 12 and the angular speed sensor 14. Also, the processing unit 20 to be described below may perform the temperature correction process in place of the signal processing unit 16, or a temperature correction function may be incorporated into the acceleration sensor 12 and the angular speed sensor 14.

The acceleration sensor 12 and the angular speed sensor 14 may output analog signals. In this case, the signal processing unit 16 may perform A/D conversion on the output signal of the acceleration sensor 12 and the output signal of the angular speed sensor 14 to generate the sensing data.

The GPS unit 50 receives a GPS satellite signal transmitted from a GPS satellite which is a type of a position measurement satellite, performs position measurement calculation using the GPS satellite signal to calculate a position and a speed (a vector including magnitude and direction) of the user in the n frame, and outputs GPS data in which time information or measurement accuracy information is attached to the position and the speed, to the processing unit 20. Also, since a method of generating the position and the speed using the GPS or a method of generating the time information is well known, a detailed description thereof will be omitted.

The geomagnetic sensor 60 detects respective geomagnetism in 3-axis directions crossing one another (ideally, orthogonal to one another), and outputs a digital signal (geomagnetic data) according to magnitudes and directions of the detected 3-axis geomagnetism. However, the geomagnetic sensor 60 may output an analog signal. In this case, the processing unit 20 may perform A/D conversion on the output signal of the geomagnetic sensor 60 to generate the geomagnetic data.

The communication unit 40 performs data communication between the output unit 70 and the reporting device 3. The communication unit 40 performs a process of receiving a command (measurement start command, measurement end command, and the like) transmitted from the reporting device 3 or the selected exercise analysis information to be output by the output unit 70 and transmitting the command or the exercise analysis information to the processing unit 20, and a process of receiving the output information during running or the running result information generated by the processing unit 20 and transmitting the output information during running or the running result information to the output unit 70 or the reporting device 3.

The processing unit 20 is configured to include, for example, a Central Processing Unit (CPU), a Digital Signal Processor (DSP), or an Application Specific Integrated Circuit (ASIC), and performs various operation processes or control processes according to various programs stored in the storage unit 30. In particular, when the processing unit 20 receives the sensing data, the GPS data, and the geomagnetic data from the inertial measurement unit 10, the GPS unit 50, and the geomagnetic sensor 60 and calculates the speed, the position, and the posture angle of the user using the data. Further, the processing unit 20 performs various operation processes using the calculated information and analyzes the exercise of the user to generate a variety of exercise analysis information to be described below. The processing unit 20 transmits some pieces of the generated exercise analysis information (the output information during running or the running result information to be described below) to the output unit 70 and the reporting device 3 via the communication unit 40. The output unit 70 converts the received exercise analysis information into perceptual information such as sound or vibration and outputs the perceptual information, and the reporting device 3 outputs the received exercise analysis information as a form of a text, an image or the like.

The storage unit 30 is configured to include, for example, a recording medium such as various IC memory of a Read Only Memory (ROM), a flash ROM or a Random Access Memory (RAM), a hard disk, a memory card, and the like.

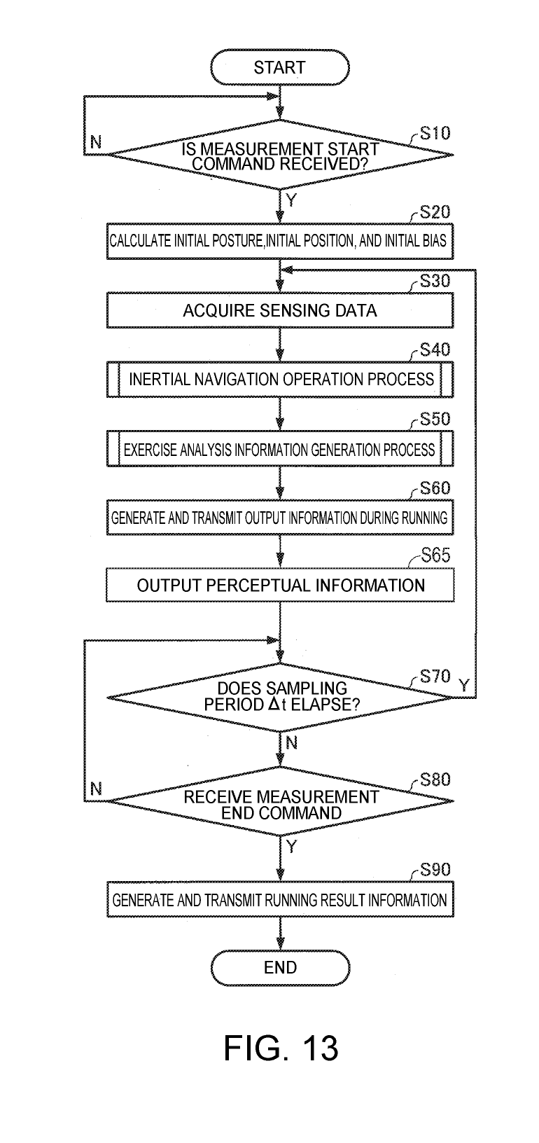

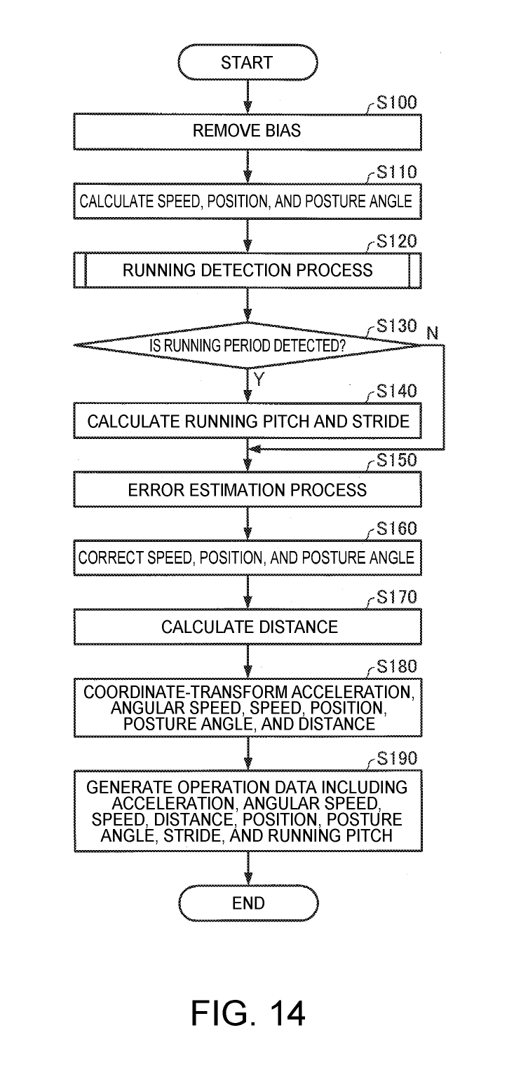

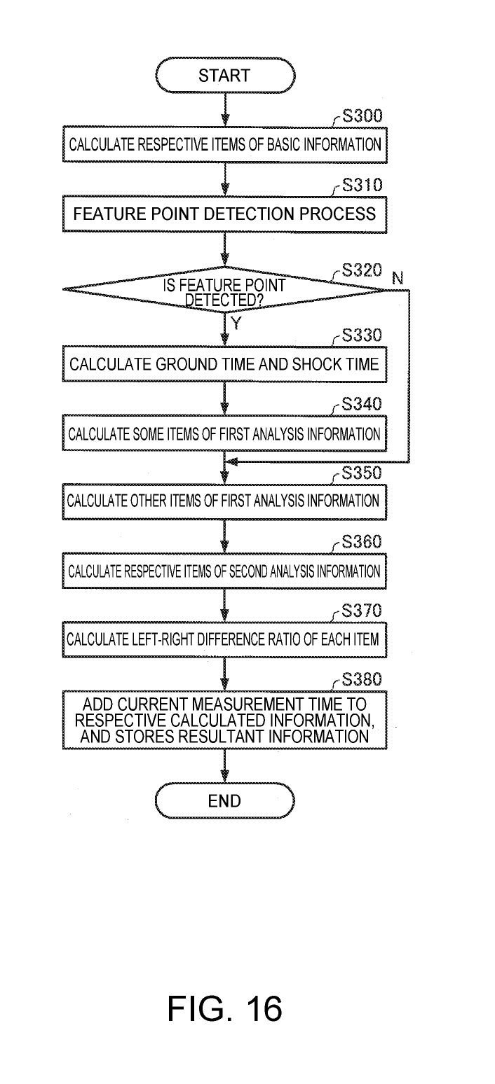

A exercise analysis program 300 read by the processing unit 20, for executing the exercise analysis process (see FIG. 13) is stored in the storage unit 30. The exercise analysis program 300 includes an inertial navigation operation program 302 for executing an inertial navigation operation process (see FIG. 14), and an exercise analysis information generation program 304 for executing the exercise analysis information generation process (see FIG. 16) as subroutines.

Further, for example, a sensing data table 310, a GPS data table 320, a geomagnetic data table 330, an operation data table 340, and exercise analysis information 350 are stored in the storage unit 30.

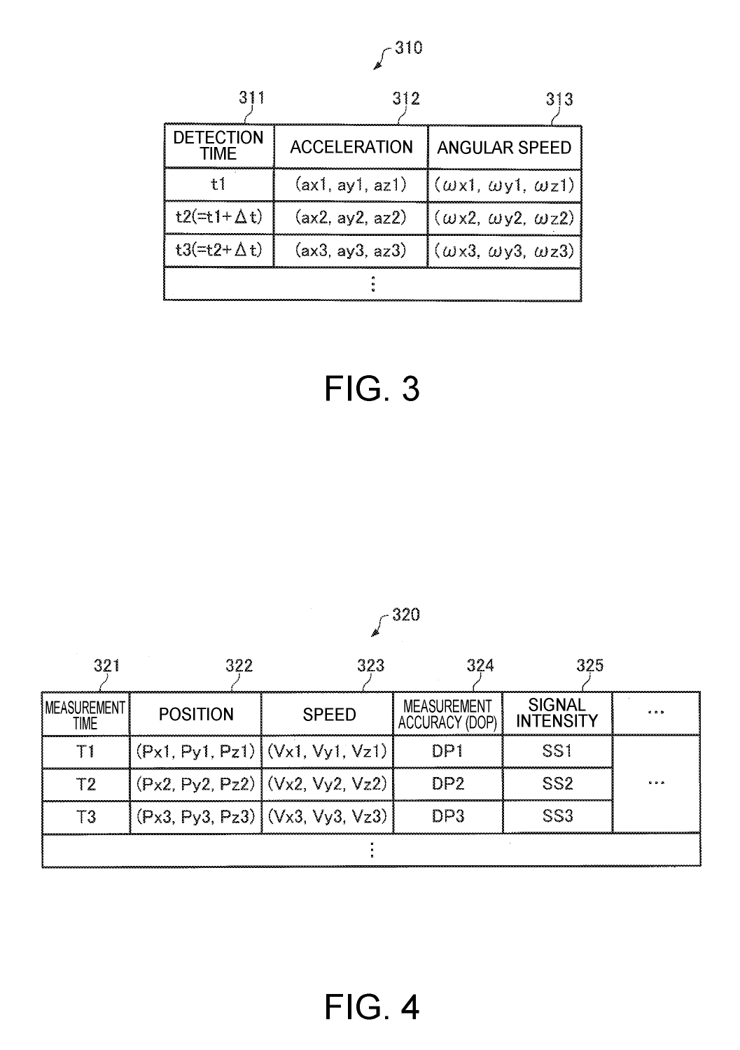

The sensing data table 310 is a data table that stores, in time series, sensing data (detection result of the inertial measurement unit 10) that the processing unit 20 receives from the inertial measurement unit 10. FIG. 3 is a diagram illustrating an example of a configuration of the sensing data table 310. As illustrated in FIG. 3, in the sensing data table 310, the sensing data in which detection time 311 of the inertial measurement unit 10, acceleration 312 detected by the acceleration sensor 12, and angular speed 313 detected by the angular speed sensor 14 are associated with one another are arranged in time series. When the measurement starts, the processing unit 20 adds new sensing data to the sensing data table 310 each time a sampling period Δt (for example, 20 ms or 10 ms) elapses. Further, the processing unit 20 corrects the acceleration and the angular speed using an acceleration bias and an angular speed bias estimated through error estimation (which will be described below) using an extended Kalman filter, and overwrites the acceleration and the angular speed after the correction to update the sensing data table 310.

The GPS data table 320 is a data table that stores, in time series, GPS data (detection result of the GPS unit (GPS sensor) 50) that the processing unit 20 receives from the GPS unit 50. FIG. 4 is a diagram illustrating an example of a configuration of the GPS data table 320. As illustrated in FIG. 4, in the GPS data table 320, GPS data in which a time 321 at which the GPS unit 50 has performed the position measurement calculation, a position 322 calculated through the position measurement calculation, speed 323 calculated through the position measurement calculation, measurement accuracy (dilution of precision; DOP) 324, signal intensity 325 of a received GPS satellite signal, and the like are associated is arranged in time series. When measurement starts, the processing unit 20 adds new GPS data to update the GPS data table 320 each time the processing unit 20 acquires the GPS data (for example, every second or asynchronously to an acquisition timing for the sensing data).

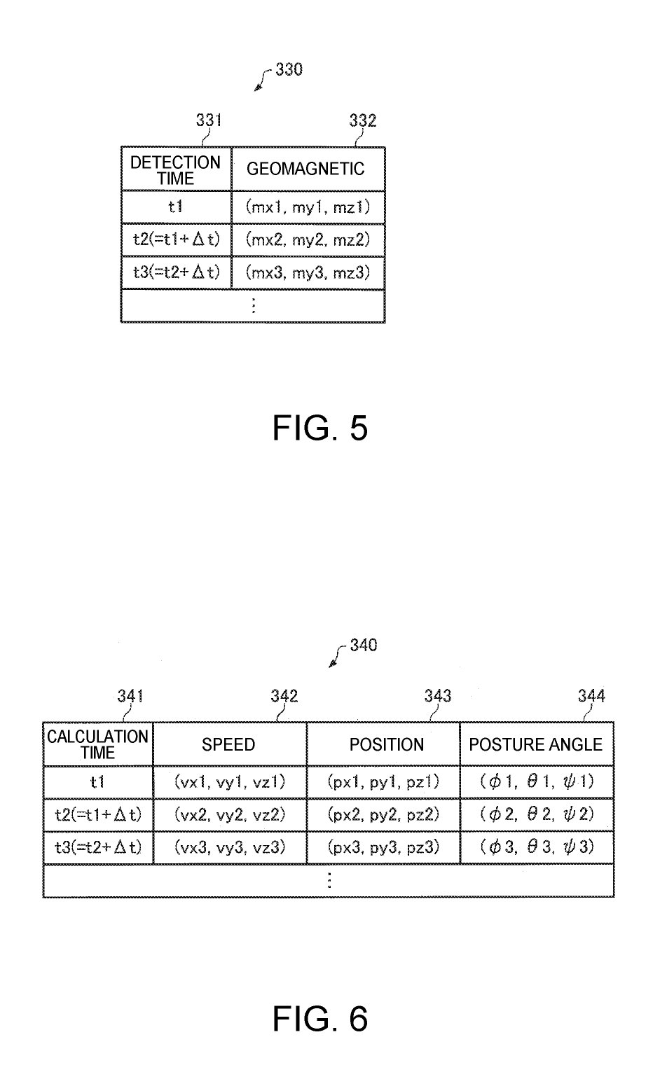

The geomagnetic data table 330 is a data table that stores, in time series, geomagnetic data (detection result of the geomagnetic sensor) that the processing unit 20 receives from the geomagnetic sensor 60. FIG. 5 is a diagram illustrating an example of a configuration of the geomagnetic data table 330. As illustrated in FIG. 5, in the geomagnetic data table 330, geomagnetic data in which detection time 331 of the geomagnetic sensor 60 and geomagnetic 332 detected by the geomagnetic sensor 60 are associated is arranged in time series. When the measurement starts, the processing unit 20 adds new geomagnetic data to the geomagnetic data table 330 each time the sampling period Δt (for example, 10 ms) elapses.

The operation data table 340 is a data table that stores, in time series, speed, a position, and a posture angle calculated using the sensing data by the processing unit 20. FIG. 6 is a diagram illustrating an example of a configuration of the operation data table 340. As illustrated in FIG. 6, in the data table 340, calculation data in which time 341 at which the processing unit 20 performs calculation, speed 342, position 343, and posture angle 344 are associated is arranged in time series. When the measurement starts, the processing unit 20 calculates the speed, the position, and the posture angle each time the processing unit 20 acquires new sensing data, that is, each time the sampling period Δt elapses, and adds new calculation data to the operation data table 340. Further, the processing unit 20 corrects the speed, position, and the posture angle using a speed error, a position error, and a posture angle error estimated through the error estimation using the extended Kalman filter, and overwrites the speed, the position, and the posture angle after the correction to update the operation data table 340.

The exercise analysis information 350 is a running state related to the exercise of the user, and includes, for example, each item of input information 351, each item of basic information 352, each item of first analysis information 353, each item of second analysis information 354, and each item of a left-right difference ratio 355 generated by the processing unit 20. Details of the information on the variety of information will be described below.

Based on data detected by the inertial measurement unit (IMU) 10, the output unit 70 receives the exercise analysis information 350 calculated by an exercise analysis unit 24, converts the exercise analysis information 350 into perceptual information, and outputs the perceptual information. For this reason, even when the user is running, since the exercise analysis information 350 of the user is converted and reported as perceptual information, it is possible to easily check the exercise analysis information 350 while the user is running. More specifically, among the exercise analysis information 350, the output unit 70 converts indexes representing a running state, which periodically occurs during running of the user, and which the user is unlikely to constantly monitor into perceptual information and outputs the perceptual information.

The perceptual information is at least one of sound, light, or vibration, and is output correspondingly to values of the exercise analysis information 350. For example, in a case where the exercise analysis information 350 is information related to a speed or an acceleration during running of the user, the exercise analysis information 350 is converted into sound, light, or vibration having a frequency corresponding to the speed or the acceleration and is output in synchronization with landing. That is, the frequency of the sound, light, or vibration is changed corresponding to magnitude of the speed or the acceleration, and the frequency is output in a comparison relationship or a reverse-comparison relationship.

In addition, in a case where the exercise analysis information 350 is information related to a stride, a pitch, propulsion efficiency, an amount of brake at the time of landing, or ground time during running of the user, the exercise analysis information 350 is not limited to sound, light, or vibration having a frequency corresponding to values of the exercise analysis information 350, but may be information on magnitudes of a time interval or a volume corresponding to values of the exercise analysis information 350. That is, sound having a constant frequency is emitted with magnitudes of the time interval or the volume corresponding to the value of the exercise analysis information 350, for example, it is possible to check the exercise analysis information 350 by emitting “beep-beep”, “pee-pee”, or the like or changing magnitudes of the volume and emitting the sound.

In addition, in a case where the perceptual information is sound, the sound may be onomatopoeia or mimetic sound obtained by voice conversion of mimetic words. For example, in a case where ground time is short during running of the user, sound “beep-beep” is emitted and in a case where ground time is long during running of the user, sound “pee-pee” is emitted, so that the sound becomes easy to hear and it is possible to accurately check the exercise analysis information 350.

In addition, the exercise analysis information 350 may be output as different perceptual information for a left foot and a right foot of the user. For example, in a case where the exercise analysis information 350 is information related to the left foot, the exercise analysis information 350 is converted into perceptual information having a low frequency bandwidth and is output, and in a case where the exercise analysis information 350 is information related to the right foot, the exercise analysis information 350 is converted into perceptual information having a high frequency bandwidth and is output. By outputting as different perceptual information for the left foot and the right foot, the user can easily determine and check whether the exercise analysis information 350 is related to the exercise analysis information 350 on the left foot or the right foot.

The exercise analysis information 350 converted into perceptual information is preferably output at a timing when the left foot or the right foot of the user during running is landed or is more preferably output within ±100 ms at the time of landing. By outputting the perceptual information at the timing, it is possible to check the exercise analysis information 350 while accurately maintaining rhythm of the user during running.

### 3-2. Functional Configuration of Processing Unit

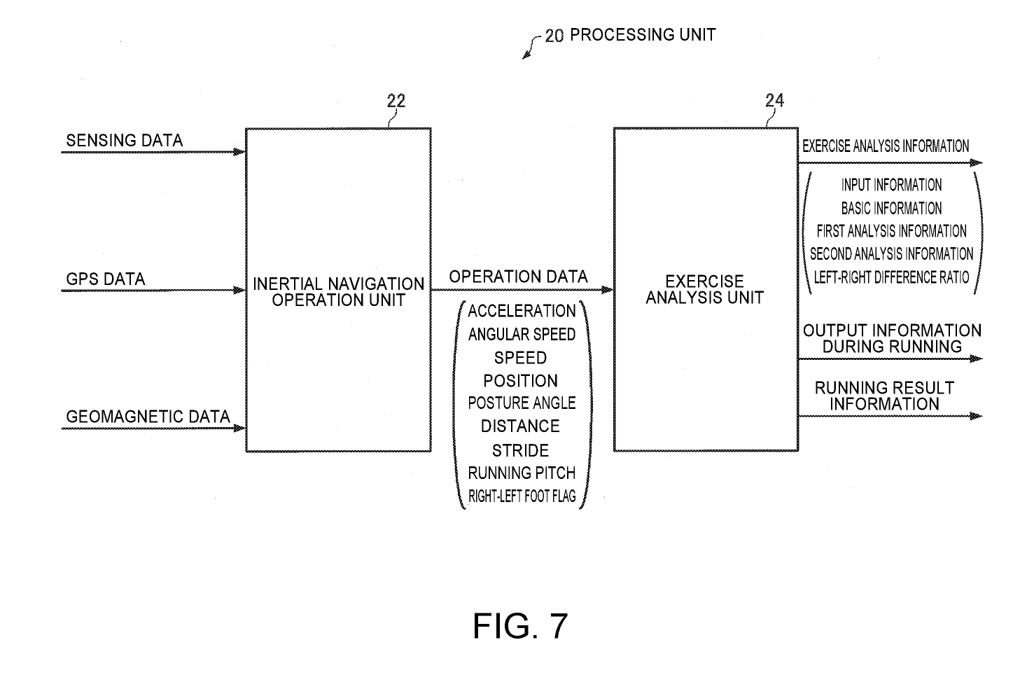

FIG. 7 is a functional block diagram illustrating an example of a configuration of the processing unit 20 of the exercise analysis device 2 of the present embodiment. In the present embodiment, the processing unit 20 executes the exercise analysis program 300 stored in the storage unit 30 to function as an inertial navigation operation unit 22 and the exercise analysis unit 24 as an exercise information generation unit.

The inertial navigation operation unit 22 performs inertial navigation calculation using the sensing data (detection result of the inertial measurement unit 10), the GPS data (detection result of the GPS unit 50), and geomagnetic data (detection result of the geomagnetic sensor 60) to calculate the acceleration, the angular speed, the speed, the position, the posture angle, the distance, the stride, and the running pitch, and outputs operation data including these calculation results. The operation data output by the inertial navigation operation unit 22 is stored in the storage unit 30. Details of the inertial navigation operation unit 22 will be described below.

The exercise analysis unit 24 as the exercise information generation unit analyzes the exercise during running of the user using the operation data (operation data stored in the storage unit 30) output by the inertial navigation operation unit 22, and generates exercise analysis information (for example, the input information 351, the basic information 352, the first analysis information 353, the second analysis information 354, and the left-right difference ratio 355 to be described below) that is information on an analysis result. The exercise analysis information generated by the exercise analysis unit 24 is stored in the storage unit 30 in time order during running of the user.

Further, the exercise analysis unit 24 generates output information during running that is information output during running of the user (specifically, between start and end of measurement in the inertial measurement unit 10) using the generated exercise analysis information. The output information during running generated by the exercise analysis unit 24 is transmitted to the output unit 70 and the reporting device 3 via the communication unit 40.

Further, the exercise analysis unit 24 generates the running result information that is information on the running result at the time of running end of the user (specifically, at the time of measurement end of the inertial measurement unit 10) using the exercise analysis information generated during running. The running result information generated by the exercise analysis unit 24 is transmitted to the reporting device 3 via the communication unit 40.

### 3-3. Functional Configuration of Inertial Navigation Operation Unit

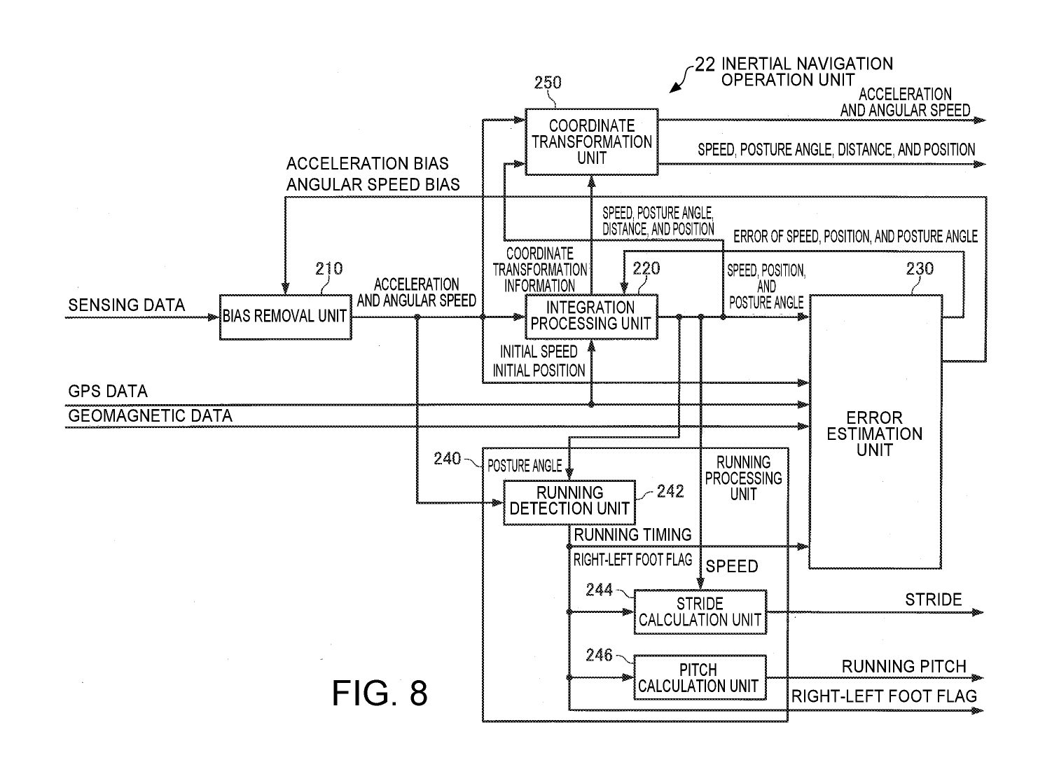

FIG. 8 is a functional block diagram illustrating an example of a configuration of the inertial navigation operation unit 22. In the present embodiment, the inertial navigation operation unit 22 includes a bias removal unit 210, an integration processing unit 220, an error estimation unit 230, a running processing unit 240, and a coordinate transformation unit 250. However, in the inertial navigation operation unit 22 of the present embodiment, some of these components may be removed or changed, or other components may be added.

The bias removal unit 210 performs a process of subtracting an acceleration bias bₐ and an angular speed bias b_{ω} estimated through the error estimation unit 230 from the 3-axis acceleration and 3-axis angular speed included in the newly acquired sensing data to correct the 3-axis acceleration and the 3-axis angular speed. Also, since there are no estimation values of the acceleration bias bₐ and the angular speed bias b_{ω} in the initial state immediately after the start of measurement, the bias removal unit 210 assumes that the initial state of the user is a resting state, and calculates the initial bias using the sensing data from the inertial measurement unit (IMU) 10.

The integration processing unit 220 performs a process of calculating speed v^{e}, position p^{e}, and a posture angle (roll angle φ_{be}, pitch angle θ_{be}, and yaw angle ψ_{be}) of the e frame from the acceleration and the angular speed corrected by the bias removal unit 210. Specifically, the integration processing unit 220 first assumes that an initial state of the user is a resting state, sets initial speed to zero, calculates the initial speed from the speed included in the GPS data, and calculates an initial position from the position included in the GPS data. Further, the integration processing unit 220 specifies a direction of the gravitational acceleration from the 3-axis acceleration of the b frame corrected by the bias removal unit 210, calculates initial values of the roll angle φ_{be} and the pitch angle θ_{be}, calculates the initial value of the yaw angle ψ_{be} from the speed included in the GPS data, and sets the initial values as an initial posture angle of the e frame. When the GPS data cannot be obtained, the initial value of the yaw angle ψ_{be} is set to, for example, zero. Also, the integration processing unit 220 calculates an initial value of a coordinate transformation matrix (rotation matrix) C_{b}^{e} from the b frame to the e frame, which is expressed as Equation (1), from the calculated initial posture angle.

$${C}_{b}^{e} = [ \begin{matrix}cos {θ}_{be} \cdot cos {ϕ}_{be} & cos {θ}_{be} \cdot sin {ϕ}_{be} & - sin {θ}_{be} \\ \begin{matrix}sin {φ}_{be} \cdot sin {θ}_{be} \cdot cos {ϕ}_{be} - \\ cos {φ}_{be} \cdot sin {ϕ}_{be}\end{matrix} & \begin{matrix}sin {φ}_{be} \cdot sin {θ}_{be} \cdot sin {ϕ}_{be} + \\ cos {φ}_{be} \cdot cos {ϕ}_{be}\end{matrix} & \begin{matrix}sin {φ}_{be} \cdot \\ cos {θ}_{be}\end{matrix} \\ \begin{matrix}cos {φ}_{be} \cdot sin {θ}_{be} \cdot cos {ϕ}_{be} + \\ sin {φ}_{be} \cdot sin {ϕ}_{be}\end{matrix} & \begin{matrix}cos {φ}_{be} \cdot sin {θ}_{be} \cdot sin {ϕ}_{be} - \\ sin {φ}_{be} \cdot cos {ϕ}_{be}\end{matrix} & \begin{matrix}cos {φ}_{be} \cdot \\ cos {θ}_{be}\end{matrix}\end{matrix} ] \qquad ( 1 )$$

Then, the integration processing unit 220 integrates the 3-axis angular speed corrected by the bias removal unit 210 (rotation operation) to calculate a coordinate transformation matrix C_{b}^{e}, and calculates the posture angle using Equation (2).

$$[ \begin{matrix}{φ}_{be} \\ {θ}_{be} \\ {ϕ}_{be}\end{matrix} ] = [ \begin{matrix}arc tan 2 ( {C}_{b}^{e} ( 2 , 3 ) , {C}_{b}^{e} ( 3 , 3 ) ) - \\ arc sin {C}_{b}^{e} ( 1 , 3 ) \\ arc tan 2 ( {C}_{b}^{e} ( 1 , 2 ) , {C}_{b}^{e} ( 1 , 1 ) )\end{matrix} ] \qquad ( 2 )$$

Further, the integration processing unit 220 converts the 3-axis acceleration of the b frame corrected by the bias removal unit 210 into the 3-axis acceleration of the e frame using the coordinate transformation matrix C_{b}^{e}, and removes and integrates a gravitational acceleration component to calculate the speed v^{e} of the e frame. Further, the integration processing unit 220 integrates the speed v^{e} of the e-frame to calculate the position p^{e} of the e frame.

Further, the integration processing unit 220 performs a process of correcting the speed v^{e}, the position p^{e}, and the posture angle using the speed error δv^{e}, the position error δp^{e}, and the posture angle error ε^{e} estimated by the error estimation unit 230, and a process of integrating the corrected speed v^{e} to calculate a distance.

Further, the integration processing unit 220 also calculates a coordinate transformation matrix C_{b}^{m} from the b frame to the m frame, a coordinate transformation matrix Cₑ^{m} from the e frame to the m frame, and a coordinate transformation matrix Cₑⁿ from the e frame to the n frame. These coordinate transformation matrixes are used as coordinate transformation information for a coordinate transformation process of the coordinate transformation unit 250 to be described below.

The error estimation unit 230 estimates an error of the index indicating the state of the user using, for example, the speed, the position, and the posture angle calculated by the integration processing unit 220, the acceleration or the angular speed corrected by the bias removal unit 210, GPS data, and the geomagnetic data. In the present embodiment, the error estimation unit 230 estimates the errors of indexes of the speed, the posture angle, the acceleration, the angular speed, and the position, which are the indexes representing a state of the user, using the extended Kalman filter. That is, the error estimation unit 230 defines the state vector X as in Equation (3) by setting the error of the speed v^{e} (speed error) δ^{e} calculated by the integration processing unit 220, the error of the posture angle (posture angle error) ε^{e} calculated by the integration processing unit 220, the acceleration bias bₐ, the angular bias b., and the error of the position p^{e} (position error) δp^{e} calculated by the integration processing unit 220, as state variables of the extended Kalman filter.

$$X = [ \begin{matrix}δ {v}^{e} \\ {ɛ}^{e} \\ {b}_{a} \\ {b}_{ω} \\ δ {p}^{e}\end{matrix} ] \qquad ( 3 )$$

The error estimation unit 230 predicts a state variable (an error between indexes representing a state of the user) included in the state vector X using a prediction equation of the extended Kalman filter. The prediction equation of the extended Kalman filter is expressed by Equation (4). In Equation (4), a matrix ϕ is a matrix that associates a previous state vector X with a current state vector X, and some of elements of the matrix are designed to change every moment while reflecting, for example, the posture angle or the position. In addition, Q is a matrix representing process noise, and each element of Q is set to an appropriate value in advance. Further, P is an error covariance matrix of the state variable.

X=ΦX

P=ΦPΦ^{T}+Q (4)

Further, the error estimation unit 230 updates (corrects) the predicted state variable (an error between indexes representing a state of the user) using the updating equation of the extended Kalman filter. The updating equation of the extended Kalman filter is expressed as Equation (5). Z and H are an observation vector and an observation matrix, respectively. The updating equation (5) shows that the state vector X is corrected using a difference between an actual observation vector Z and a vector HX predicted from the state vector X. R is a covariance matrix of the observation error, and may be a predetermined constant value or may be dynamically changed. K indicates a Kalman gain, and K increases as R decreases. By Equation (5), as K increases (R decreases), an amount of correction of the state vector X increases and P correspondingly decreases.

K=PH^{T}(HPH^{T}+R)⁻¹

X=X+K(Z−HX)

P=(I−KH)P (5)

Examples of an error estimation method (method of estimating the state vector X) include the following methods.

### Error Estimation Method Using Correction Based on Posture Angle Error

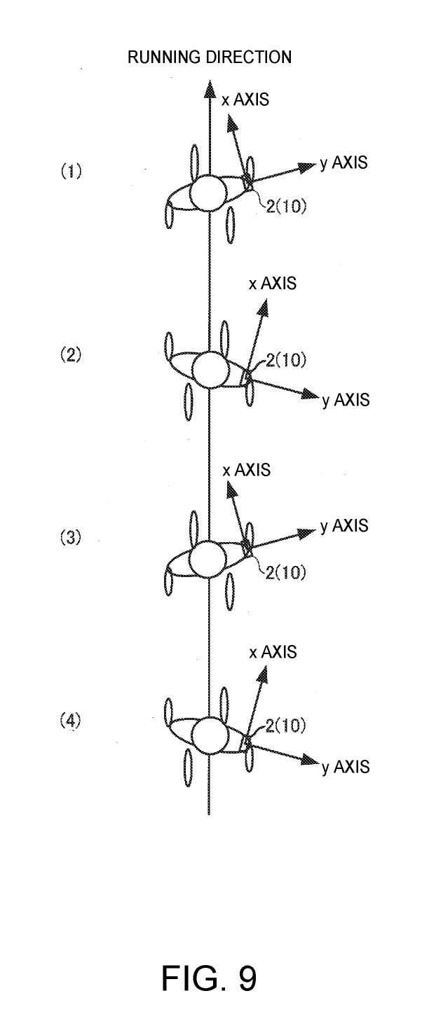

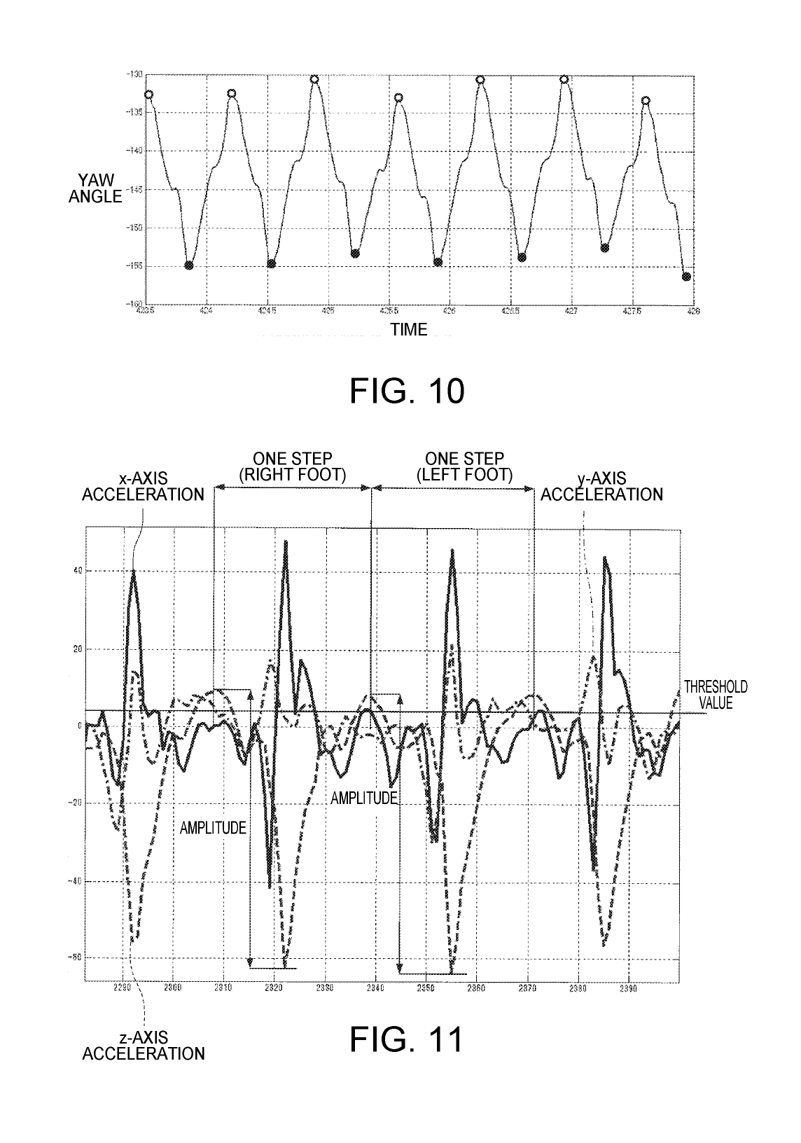

FIG. 9 is a diagram illustrating an overhead view of the exercise of the user when a user wearing the exercise analysis device 2 on the right waist performs a running operation (straight running). Further, FIG. 10 is a diagram illustrating an example of a yaw angle (azimuth angle) calculated from the detection result of the inertial measurement unit 10 when the user performs a running operation (straight running). A horizontal axis indicates time and a vertical axis indicates a yaw angle (azimuth angle).

With the running operation of the user, the posture of the inertial measurement unit 10 with respect to the user changes at any time. In a state in which the user steps forward with a left foot, the inertial measurement unit 10 has a posture inclined to the left with respect to the running direction (x axis of the m frame), as illustrated in (1) or (3) in FIG. 9. On the other hand, in a state in which the user steps forward with a right foot, the inertial measurement unit 10 has a posture inclined to the right side with respect to the running direction (x axis of the m frame) as illustrated in (2) or (4) in FIG. 9. That is, the posture of the inertial measurement unit 10 periodically changes in every two steps of one left step and one right step with the running operation of the user. In FIG. 10, for example, the yaw angle is maximized in a state in which the user steps forward with the right foot (∘ in FIG. 10), and the yaw angle is minimized in a state in which the user steps forward with the left foot (⋅ in FIG. 10). Therefore, the error can be estimated on the assumption that a previous (before two steps) posture angle and a current posture angle are equal, and the previous posture angle is a true posture. In this method, the observation vector Z in Equation (5) is a difference between the previous posture angle and the current posture angle calculated by the integration processing unit 220, and the state vector X is corrected based on a difference between the posture angle error ε^{e} and the observation value using the updating equation (5), and the error is estimated.

### Error Estimation Method Using Correction Based on Angular Speed Bias

This is a method of estimating the error on the assumption that a previous (before two steps) posture angle is equal to the current posture angle, but it is not necessary for the previous posture angle to be a true posture. In this method, the observation vector Z in Equation (5) is an angular speed bias calculated from the previous posture angle and the current posture angle calculated by the integration processing unit 220. Using the updating equation (5), the state vector X is corrected based on a difference between the angular speed bias b_{ω} and the observation value, and the error is estimated.

### Error Estimation Method Using Correction Based on Azimuth Angle Error

This is a method of estimating the error on the assumption that a previous (before two steps) yaw angle (azimuth angle) is equal to a current yaw angle (azimuth angle), and the previous yaw angle (azimuth angle) is a true yaw angle (azimuth angle). In this method, the observation vector Z is a difference between the previous yaw angle and the current yaw angle calculated by the integration processing unit 220. Using the updating equation (5), the state vector X is corrected based on a difference between the azimuth angle error ε_{z}^{e} and the observation value, and the error is estimated.

### Error Estimation Method Using Correction Based on Stop

This is a method of estimating the error on the assumption that the speed is zero at the time of stop. In this method, the observation vector Z is a difference between the speed v^{e} calculated by the integration processing unit 220 and zero. Using the updating equation (5), the state vector X is corrected based on the speed error δv^{e}, and the error is estimated.

### Error Estimation Method Using Correction Based on Rest

This is a method of estimating the error on the assumption that the speed is zero at rest, and a posture change is zero. In this method, the observation vector Z is an error of the speed ve calculated by the integration processing unit 220, and a difference between the previous posture angle and the current posture angle calculated by the integration processing unit 220. Using the updating equation (5), the state vector X is corrected based on the speed error δve and the posture angle error εe, and the error is estimated.

### Error Estimation Method Using Correction Based on GPS Observations Value

This is a method of estimating the error on the assumption that the speed v^{e}, the position p^{e}, or the yaw angle ψ_{be} calculated by the integration processing unit 220 is equal to the speed, position, or azimuth angle (the speed, position, or azimuth angle after conversion into the e frame) calculated from the GPS data. In this method, the observation vector Z is a difference between the speed, position, or yaw angle calculated by the integration processing unit 220 and the speed, position, or azimuth angle calculated from the GPS data. Using the updating equation (5), the state vector X is corrected based on a difference between the speed error δv^{e}, the position error δp^{e}, or the azimuth angle error ε_{z}^{e} and the observation value, and the error is estimated.

### Error Estimation Method Using Correction Based on Observation Value of Geomagnetic Sensor

This is a method of estimating the error on the assumption that the yaw angle ψ_{be} calculated by the integration processing unit 220 is equal to the azimuth angle (azimuth angle after conversion into the e-frame) calculated from the geomagnetic sensor 60. In this method, the observation vector Z is a difference between the yaw angle calculated by the integration processing unit 220 and the azimuth angle calculated from the geomagnetic data. Using the updating equation (5), the state vector X is corrected based on the difference between the azimuth angle error ε_{z}^{e} and the observation value, and the error is the estimated.

Referring back to FIG. 8, the running processing unit 240 includes a running detection unit 242, a stride calculation unit 244, and a pitch calculation unit 246. The running detection unit 242 performs a process of detecting the running period (running timing) of the user using a detection result of the inertial measurement unit 10 (specifically, the sensing data corrected by the bias removal unit 210). As described in FIGS. 9 and 10, since the posture of the user changes periodically (every two steps (one left step and one right step)) at the time of running of the user, the acceleration detected by the inertial measurement unit 10 also changes periodically. FIG. 11 is a diagram illustrating an example of a 3-axis acceleration detected by the inertial measurement unit 10 at the time of running of the user. In FIG. 11, a horizontal axis indicates time and a vertical axis indicates an acceleration value. As illustrated in FIG. 11, the 3-axis acceleration periodically changes, and in particular, the z-axis (axis in a direction of gravity) acceleration can be seen as regularly changing with a periodicity. This z-axis acceleration reflects the acceleration of a vertical movement of the user, and a period from a time at which the z-axis acceleration becomes a maximum value equal to or greater than a predetermined threshold value to a time at which the z-axis acceleration next becomes the maximum value equal to or greater than the threshold value corresponds to a period of one step. One step of stepping on the right foot and one step of stepping on the left foot are alternately repeated.

Therefore, in the present embodiment, the running detection unit 242 detects the running period each time the z-axis acceleration (corresponding to the acceleration of the vertical movement of the user) detected by the inertial measurement unit 10 becomes the maximum value equal to or greater than the predetermined threshold value. That is, the running detection unit 242 outputs a timing signal indicating that the running detection unit 242 detects the running period each time the z-axis acceleration becomes the maximum value equal to or greater than the predetermined threshold value. In fact, since a high-frequency noise component is included in the 3-axis acceleration detected by the inertial measurement unit 10, the running detection unit 242 detects the running period using the z-axis acceleration passing through a low pass filter so that noise is removed.

Further, the running detection unit 242 determines whether the detected running period is a left running period or a right running period, and outputs a right-left foot flag (for example, ON for the right foot and OFF for left foot) indicating whether the detected running period is a left running period or a right running period. For example, as illustrated in FIG. 10, since the yaw angle is maximized (∘ in FIG. 10) in a state in which the right leg steps forward, and the yaw angle is minimized (● in FIG. 10) in a state in which the left leg steps forward, the running detection unit 242 can determine whether the running cycle is a left running cycle or a right running cycle using the posture angle (particularly, the yaw angle) calculated by the integration processing unit 220. Further, as illustrated in FIG. 9, when viewed from an overhead side of the user, the inertial measurement unit 10 rotates clockwise from a state in which the user steps forward with the left foot (state (1) or (3) in FIG. 9) to a state in which the user steps forward with the right foot (state (2) or (4) in FIG. 9), and reversely rotates in a counterclockwise from the state in which the user steps forward with the right foot to the state in which the user steps forward with the left foot. Thus, for example, the running detection unit 242 can also determine whether the running period is a left running period or a right running period based on a polarity of the z-axis angular speed. In this case, since a high-frequency noise component is, in fact, included in the 3-axis angular speed detected by the inertial measurement unit 10, the running detection unit 242 determines whether the running period is a left running period or a right running period using the z-axis angular speed passing through a low pass filter so that noise is removed.

There is a case where the user does not know which one of the right foot and the left foot will start running, or it may fail to detect the running cycle during running, so that the running detection unit 242 may synthetically determine whether the running period is the running period of the right foot or the running period of the left foot using information (for example, posture angle or the like) other than the z-axis acceleration.

The stride calculation unit 244 performs a process of calculating right and left strides using a timing signal of the running period output by the running detection unit 242, the right-left foot flag, and the speed or the position calculated by the integration processing unit 220, and outputs the strides as right and left strides. That is, the stride calculation unit 244 integrates the speed in a period from the start of the running period to the start of the next running period at every sampling period Δt (or calculates a difference between a position at the time of start of the running period and a position at the time of start of the next running period) to calculate the stride, and outputs the stride as a stride.

The pitch calculation unit 246 performs a process of calculating the number of steps for 1 minute using the timing signal having a running period output by the running detection unit 242, and outputting the number of steps as a running pitch. That is, the pitch calculation unit 246, for example, takes a reciprocal of the running period to calculate the number of steps per second, and multiplies the number of steps by 60 to calculate the number of steps (running pitch) for 1 minute.

The coordinate transformation unit 250 performs a coordinate transformation process of transforming the 3-axis acceleration and the 3-axis angular speed of the b frame corrected by the bias removal unit 210 into the 3-axis acceleration and the 3-axis angular speed of the m frame using the coordinate transformation information (coordinate transformation matrix C_{b}^{m}) from the b frame to the m-frame calculated by the integration processing unit 220. Further, the coordinate transformation unit 250 performs a coordinate transformation process of transforming the speed in the 3-axis direction, the posture angle around the 3-axis direction, and the distance in the 3-axis direction of the e frame calculated by the integration processing unit 220 into the speed in the 3-axis direction, the posture angle around the 3-axis direction, and the distance in the 3-axis direction of the m frame using the coordinate transformation information (coordinate transformation matrix Cₑ^{m}) from the e frame to the m-frame calculated by the integration processing unit 220. Further, the coordinate transformation unit 250 performs a coordinate transformation process of transforming a position of the e frame calculated by the integration processing unit 220 into a position of the n frame using the coordinate transformation information (coordinate transformation matrix Cₑⁿ) from the e frame to the n frame calculated by the integration processing unit 220.

Also, the inertial navigation operation unit 22 outputs operation data including respective information of the acceleration, the angular speed, the speed, the position, the posture angle, and the distance after coordinate transformation in the coordinate transformation unit 250, and the stride, the running pitch, and right-left foot flags calculated by the running processing unit 240 (stores the information in the storage unit 30).

### 3-4. Functional Configuration of Exercise Analysis Device

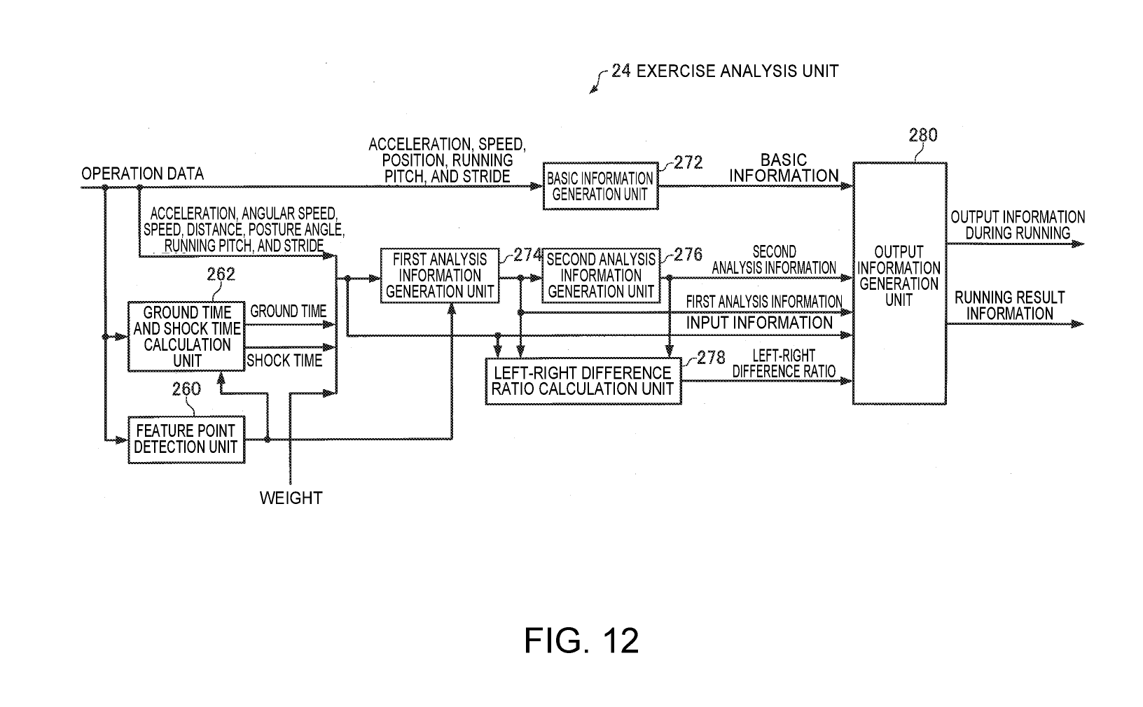

FIG. 12 is a functional block diagram illustrating an example of a configuration of the exercise analysis unit 24 in the present embodiment. In the present embodiment, the exercise analysis unit 24 includes a feature point detection unit 260, a ground time and shock time calculation unit 262, a basic information generation unit 272, a first analysis information generation unit 274, a second analysis information generation unit 276, a left-right difference ratio calculation unit 278, and an output information generation unit 280. However, in the exercise analysis unit 24 of the present embodiment, some of these components may be removed or changed, or other components may be added.

The feature point detection unit 260 performs a process of detecting a feature point in the running exercise of the user using the operation data. Examples of the feature point in the running exercise of the user includes landing (for example, a time when a portion of a sole of the foot arrives at the ground, a time when the entire sole of the foot arrives on the ground, any time point while a heel of the foot first arrives and then a toe thereof is separated, any time point while the toe of the foot first arrives and then the heel thereof is separated, and a time while the entire sole of the foot arrives may be appropriately set), depression (a state which most weight is applied to the foot), and separation from ground (also referred to as kicking; a time when a portion of the sole of the foot is separated from the ground, a time when the entire sole of the foot is separated from the ground, any time point while a heel of the foot first arrives and then a toe thereof is separated, and any time point while the toe of the foot first arrives and then is separated may be appropriately set). Specifically, the feature point detection unit 260 separately detects the feature point in the running period of the right food and the feature point in the running period of the left foot using the right-left foot flag included in the operation data. For example, the feature point detection unit 260 can detect the landing at a timing at which the acceleration in the vertical direction (detection value of the z axis of the acceleration sensor 12) changes from a positive value to a negative value, detect depression at a time point at which the acceleration in a running direction becomes a peak after the acceleration in the vertical direction becomes a peak in a negative direction after landing, and detect separation from ground (kicking) at a time point at which the acceleration in the vertical direction changes from a negative value to a positive value.

The ground time and shock time calculation unit 262 performs a process of calculating respective values of the ground time and the shock time based on a timing as a reference at which the feature point detection unit 260 detects the feature point using the operation data. Specifically, the ground time and shock time calculation unit 262 determines whether current operation data is operation data of the running period of the right foot or operation data of the running period of the left foot from the right-left foot flag included in the operation data, and calculates the respective values of the ground time and the shock time in the running period of the right foot and the running period of the left foot based on a time point at which the feature point detection unit 260 detects the feature point. Definitions, calculation methods, and the like of the ground time and the shock time will be described below in detail.

The basic information generation unit 272 performs a process of generating basic information 352 on the exercise of the user using the information on the acceleration, speed, position, stride, and running pitch included in the operation data. Here, the basic information 352 includes respective items of the running pitch, the stride, the running speed, altitude, running distance, and running time (lap time). Specifically, the basic information generation unit 272 outputs the running pitch and the stride included in the operation data as the running pitch and the stride of the basic information 352. Further, the basic information generation unit 272 calculates the exercise analysis information, for example, current values of the running speed, the altitude, the running distance, and the running time (lap time) or average values thereof during running using some or all of the acceleration, the speed, the position, the running pitch, and the stride included in the operation data.

The first analysis information generation unit 274 analyzes user's exercise based on a timing at which the feature point detection unit 260 detects the feature point using the input information 351, and performs a process of generating the first analysis information 353.

Here, the input information 351 includes respective items of acceleration in a running direction, speed in the running direction, distance in the running direction, acceleration in the vertical direction, speed in the vertical direction, distance in the vertical direction, acceleration in a horizontal direction, horizontal direction speed, distance in the horizontal direction, posture angle (roll angle, pitch angle, and yaw angle), angular speed (roll direction, pitch direction, and yaw direction), running pitch, stride, ground time, shock time, and weight. The body weight is input by the user, ground time and shock time are calculated by the ground time and shock time calculation unit 262, and other items are included in the operation data.

Further, the first analysis information 353 includes respective items of amounts of brake at the time of landing (amount of brake 1 at the time of landing, and amount of brake at the time of landing), directly-under landing rates (directly-under landing rate 1, directly-under landing rate 2, and directly-under landing rate 3), propulsion power (propulsion power 1, and propulsion power 2), propulsion efficiency (propulsion efficiency 1, propulsion efficiency 2, propulsion efficiency 3, and propulsion efficiency 4), an amount of energy consumption, landing shock, running capability, an anteversion angle, and a degree of timing matching. Each item of the first analysis information 353 is an item indicating a running state (an example of an exercise state) of the user and is one piece of running information. Contents and a calculation method for each item of the first analysis information 353 will be described below in detail.

Further, the first analysis information generation unit 274 calculates the value of each item of the first analysis information 353 for left and right of the body of the user. Specifically, the first analysis information generation unit 274 calculates each item included in the first analysis information 353 in the running period of the right foot and the running period of the left foot according to whether the feature point detection unit 260 detects the feature point in the running period of the right foot or the feature point in the running period of the left foot. Further, the first analysis information generation unit 274 also calculates left and right average values or a sum value for each item included in the first analysis information 353.

The second analysis information generation unit 276 performs a process of generating the second analysis information 354 using the first analysis information 353 generated by the first analysis information generation unit 274. Here, the second analysis information 354 includes respective items of energy loss, energy efficiency, and a load on the body. Content and a calculation method for each item of the second analysis information 354 will be described below in detail. The second analysis information generation unit 276 calculates values of the respective items of the second analysis information 354 in the running period of the right foot and the running period of the left foot. Further, the second analysis information generation unit 276 also calculates the left and right average values or the sum value for each item included in the second analysis information 354.

The left-right difference ratio calculation unit 278 performs a process of calculating the left-right difference ratio 355 that is an index indicating left-right balance of the body of the user using a value in the running period of the right foot and a value in the running period of the left foot for the running pitch, the stride, the ground time, and the shock time included in the input information 351, all items of the first analysis information 353, and all items of the second analysis information 354. The left-right difference ratio 355 for each item is one piece of exercise analysis information. Contents and a calculation method for the left-right difference ratio 355 will be described below in detail.

The output information generation unit 280 performs a process of generating the output information during running that is information output during running of the user using, for example, the basic information 352, the input information 351, the first analysis information 353, the second analysis information 354, and the left-right difference ratio 355. “Running pitch”, “stride”, “ground time”, and “shock time” included in the input information 351, all items of the first analysis information 353, all items of the second analysis information 354, and the left-right difference ratio 355 are exercise indexes used for evaluation of the running skill of the user, and the output information during running includes information on values of some or all of the exercise indexes. The exercise indexes included in the output information during running may be determined in advance, or may be selected by the user operating the reporting device 3. Further, the output information during running may include some or all of running speed, altitude, a running distance, and a running time (lap time) included in the basic information 352.

Further, the output information generation unit 280 generates running result information that is information on a running result of the user using, for example, the basic information 352, the input information 351, the first analysis information 353, the second analysis information 354, and the left-right difference ratio 355. For example, the output information generation unit 280 may generate the running result information including, for example, information on an average value of each exercise index during running of the user (during measurement of the inertial measurement unit 10). Further, the running result information may include some or all of the running speed, the altitude, the running distance, and the running time (lap time).

The output information generation unit 280 transmits the output information during running to the reporting device 3 and the output unit 70 via the communication unit 40 during running of the user, and transmits the running result information to the reporting device 3 at the time of running end of the user.

### 3-5. Input Information

Hereinafter, respective items of input information 351 will be described in detail.

### Acceleration in Running Direction, Acceleration in Vertical Direction, and Acceleration in Horizontal Direction

A “running direction” is a running direction of the user (x-axis direction of the m frame), a “vertical direction” is a vertical direction (z-axis direction of the m frame), and a “horizontal direction” is a direction (y-axis direction of the m frame) perpendicular to the running direction and the vertical direction. The acceleration in the running direction, the acceleration in the vertical direction, and the acceleration in the horizontal direction are acceleration in the x-axis direction, acceleration in the z-axis direction, and acceleration in the y-axis direction of the m frame, respectively, and are calculated by the coordinate transformation unit 250.

### Speed in Running Direction, Speed in Vertical Direction, and Speed in Horizontal Direction

Speed in a running direction, speed in a vertical direction, and speed in a horizontal direction are speed in an x-axis direction, speed in a z-axis direction, and speed in a y-axis direction of the m frame, respectively, and are calculated by the coordinate transformation unit 250. Alternatively, acceleration in the running direction, acceleration in a vertical direction, and acceleration in a horizontal direction can be integrated to calculate the speed in the running direction, the speed in the vertical direction, and the speed in the horizontal direction, respectively.

### Angular Speed (Roll Direction, Pitch Direction, and Yaw Direction)

Angular speed in a roll direction, angular speed in a pitch direction, and angular speed in a yaw direction are angular speed around an x-axis direction, angular speed around a y-axis direction, and angular speed around a z-axis direction of the m frame, respectively, and are calculated by the coordinate transformation unit 250.

### Posture Angle (Roll Angle, Pitch Angle, and Yaw Angle)

An roll angle, a pitch angle, and a yaw angle are a posture angle around an x-axis direction, a posture angle around a y-axis direction, and a posture angle around a z-axis direction of the m frame that are output by the coordinate transformation unit 250 and are calculated by the coordinate transformation unit 250, respectively. Alternatively, an angular speed in the roll direction, an angular speed in the pitch direction, and the angular speed in the yaw direction can be integrated (rotation operation) to calculate the roll angle, the pitch angle, and the yaw angle.

### Distance in Running Direction, Distance in Vertical Direction, Distance in Horizontal Direction

A distance in the running direction, a distance in the vertical direction, and a distance in the horizontal direction are a movement distance in the x-axis direction, a movement distance in the z-axis direction, and a movement distance in the y-axis direction of them frame from a desired position (for example, a position immediately before the user starts running), respectively, and are calculated by the coordinate transformation unit 250.

### Running Pitch

A running pitch is an exercise index defined as the number of steps per minute and is calculated by the pitch calculation unit 246. Alternatively, the running pitch can be calculated by dividing the distance in the running direction for one minute by the stride.

### Stride

The stride is an exercise index defined as a stride of one step, and is calculated by the stride calculation unit 244. Alternatively, the stride can be calculated by dividing the distance in the running direction for one minute by the running pitch.

### Ground Time

A ground time is an exercise index defined as a time taken from landing to separation from ground (kicking), and is calculated by the ground time and shock time calculation unit 262. The separation from ground (kicking) is a time when the toe is separated from the ground. Also, since the ground time has high correlation with the running speed, the ground time can also be used as the running capability of the first analysis information 353.

### Shock Time

A shock time is an exercise index defined as a time at which shock generated due to landing is applied to the body, and is calculated by the ground time and shock time calculation unit 262. The shock time can be calculated as shock time=(time at which acceleration in a running direction in one step is minimized−time of landing).

### Weight

A weight is a weight of the user, and a numerical value of the weight is input by the user operating an operation unit (not illustrated) of the reporting device 3 before running.

### 3-6. First Analysis Information

Hereinafter, respective items of the first analysis information 353 calculated by the first analysis information generation unit 274 will be described in detail.

### Amount of Brake 1 at Time of Landing

An amount of brake 1 at the time of landing is an exercise index defined as an amount of speed decreased due to landing, and can be calculated as an amount of brake 1 at the time of landing=(speed in the running direction before landing−minimum speed in the running direction after landing). The speed in the running direction is decreased due to landing, and a lowest point of the speed in the running direction after landing in one step is the lowest speed in the running direction.

### Amount of Brake 2 at Time of Landing

The amount of brake 2 at the time of landing is an exercise index defined as an amount of lowest acceleration in a negative running direction generated due to landing, and matches minimum acceleration in the running direction after landing in one step. The lowest point of the acceleration in the running direction after landing in one step is the lowest acceleration in the running direction.

### Directly-Under Landing Rate 1

A directly-under landing rate 1 is an exercise index indicating whether the user can land directly under the body. When the user can land directly under the body, the amount of brake decreases and the user can efficiently run. Since the amount of brake normally increases according to the speed, the amount of brake is an insufficient index, but since the directly-under landing rate 1 is an index expressed at a rate, the same evaluation is possible according to the directly-under landing rate 1 even when the speed changes. When α=arc tan (acceleration in a running direction at the time of landing/acceleration in a vertical direction at the time of landing) using the acceleration in the running direction (negative acceleration) and the acceleration in the vertical direction at the time of landing, directly-under landing rate 1 can be calculated as directly-under landing rate 1=cos α×100 (%). Alternatively, an ideal angle α′ can be calculated using data of a plurality of persons who fast run, and directly-under landing rate 1 can be calculated as directly-under landing rate 1={1−|(α′−α)/α′|}×100 (%).

### Directly-Under Landing Rate 2

A directly-under landing rate 2 is an exercise index indicating whether the user can land directly under the body, using a degree of speed decrease, and is calculated as directly-under landing rate 2=(minimum speed in the running direction after landing/speed in the running direction directly before landing)×100 (%).

### Directly-Under Landing Rate 3

A directly-under landing rate 3 is an exercise index indicating whether the user can land directly under the body using a distance or time from landing to the foot coming directly under the body. The directly-under landing rate 3 can be calculated as directly-under landing rate 3=(distance in the running direction when the foot comes directly under the body−distance in the running direction at the time of landing), or as directly-under landing rate 3=(time when the foot comes directly under the body−time of landing). After landing (point at which the acceleration in the vertical direction is changed from a positive value to a negative value), there is a timing at which the acceleration in the vertical direction becomes a peak in a negative direction, and this time can be determined to be a timing (time) at which the foot comes directly under the body.

Also, in addition, the directly-under landing rate 3 may be defined as directly-under landing rate 3=β=arc tan (distance from landing to the foot coming directly under the body/height of waist). Alternatively, the directly-under landing rate 3 may be defined as directly-under landing rate 3=(1−distance from landing to the foot coming directly under the body/distance of movement from landing to kicking)×100 (%) (a ratio of the distance from landing to the foot coming directly under the body to a distance of movement while the foot is grounded). Alternatively, the directly-under landing rate 3 may be defined as directly-under landing rate 3=(1−time from landing to the foot coming directly under the body/time of movement from landing to kicking)×100 (%) (a ratio of the time from landing to the foot coming directly under the body to time of movement while the foot is grounded).

### Propulsion Power 1

Propulsion power 1 is an exercise index defined as amount of speed increasing in the running direction by kicking the ground, and can be calculated using propulsion power 1=(maximum speed in running direction after kicking−minimum speed in running direction before kicking).

### Propulsion Power 2

Propulsion power 2 is an exercise index defined as maximum acceleration in a positive running direction generated by kicking, and matches maximum acceleration in the running direction after kicking in one step.

### Propulsion Efficiency 1

Propulsion efficiency 1 is an exercise index indicating whether kicking power efficiently becomes propulsion power. When wasteful vertical movement and wasteful horizontal movement disappear, efficient running is possible. Typically, since the vertical movement and the horizontal movement increase according to the speed, the vertical movement and the horizontal movement are insufficient as indexes, but since propulsion efficiency 1 is the index expressed at a rate, the same evaluation is possible according to propulsion efficiency 1 even when the speed changes. The propulsion efficiency 1 is calculated in each of the vertical direction and the horizontal direction. When γ=arc tan (acceleration in the vertical direction at the time of kicking/acceleration in a running direction at the time of kicking) using the acceleration in the vertical direction and the acceleration in a running direction at the time of kicking, propulsion efficiency 1 in the vertical direction can be calculated as the propulsion efficiency 1 in the vertical direction=cos γ×100 (%). Alternatively, an ideal angle γ′ can be calculated using data of a plurality of persons who fast run, and propulsion efficiency 1 in the vertical direction can also be calculated as propulsion efficiency 1 in the vertical direction={1−|(γ′−γ)/γ′|}×100 (%). Similarly, when δ=arc tan (acceleration in a horizontal direction at the time of kicking/acceleration in a running direction at the time of kicking) using the acceleration in a horizontal direction and the acceleration in a running direction at the time of kicking, propulsion efficiency 1 in the horizontal direction can be calculated as the propulsion efficiency 1 in the horizontal direction=cos δ×100 (%). Alternatively, an ideal angle δ′ can be calculated using data of a plurality of persons who fast run, and propulsion efficiency 1 in the horizontal direction can be calculated as propulsion efficiency 1 in a horizontal direction={1−|(δ′−δ)/δ′|}×100 (%).

Also, in addition, the propulsion efficiency 1 in the vertical direction can also be calculated by replacing y with arc tan (speed in the vertical direction at the time of kicking/speed in the running direction at the time of kicking). Similarly, the propulsion efficiency 1 in the horizontal direction can also be calculated by replacing δ with arc tan (speed in the horizontal direction at the time of kicking/speed in the running direction at the time of kicking).

### Propulsion Efficiency 2

Propulsion efficiency 2 is an exercise index indicating whether the kicking power efficiently becomes propulsion power, using an angle of the acceleration at the time of depression. When ξ=arc tan (acceleration in the vertical direction at the time of depression/acceleration in a running direction at the time of depression) using the acceleration in the vertical direction and the acceleration in a running direction at the time of depression, propulsion efficiency 2 in the vertical direction can be calculated as the propulsion efficiency 2 in the vertical direction=cos ξ×100 (%). Alternatively, an ideal angle can be calculated using data of a plurality of persons who fast run, and propulsion efficiency 2 in the vertical direction can also be calculated as propulsion efficiency 2 in the vertical direction={1−|(ξ′−ξ)/ξ′|}×100 (%). Similarly, when η=arc tan (acceleration in a horizontal direction at the time of depression/acceleration in a running direction at the time of depression) using the acceleration in a horizontal direction and the acceleration in a running direction at the time of depression, propulsion efficiency 2 in the horizontal direction can be calculated as the propulsion efficiency 2 in the horizontal direction=cos η×100 (%). Alternatively, an ideal angle η′ can be calculated using data of a plurality of persons who fast run, and propulsion efficiency 2 in the horizontal direction can be calculated as propulsion efficiency 2 in a horizontal direction={1−|(η′−η)/η′|}×100 (%).

Also, in addition, propulsion efficiency 2 in the vertical direction can be calculated by replacing with arc tan (speed in the vertical direction at the time of depression/speed in the running direction at the time of depression). Similarly, propulsion efficiency 2 in the horizontal direction can also be calculated by replacing η with arc tan (speed in the horizontal direction at the time of depression/speed in the running direction at the time of depression).

### Propulsion Efficiency 3

Propulsion efficiency 3 is an exercise index indicating whether the kicking power efficiently becomes propulsion, using a jump angle. When a highest arrival point in a vertical direction in one step (½ of amplitude of a distance in the vertical direction) is H and a distance in the running direction from kicking to landing is X, propulsion efficiency 3 can be calculated using Equation (6).

$$Propulsion Efficiency 3 = arc sin ( \sqrt{\frac{16 {H}^{2}}{{X}^{2} + 16 {H}^{2}}} ) \qquad ( 6 )$$

### Propulsion Efficiency 4

Propulsion efficiency 4 is an exercise index indicating whether the kicking power efficiently becomes propulsion power using a ratio of energy used to advance in the running direction to total energy generated in one step. The propulsion efficiency 4 is calculated as propulsion efficiency 4=(energy used to advance in the running direction/energy used for one step)×100 (%). This energy is a sum of potential energy and kinetic energy.

### Amount of Energy Consumption

An amount of energy consumption is an exercise index defined as an amount of energy consumed by one-step advance, and also indicates integration in the running period of an amount of energy consumed by one-step advance. The amount of energy consumption is calculated as an amount of energy consumption=(amount of energy consumption in the vertical direction+amount of energy consumption in the running direction+amount of energy consumption in the horizontal direction). Here, the amount of energy consumption in the vertical direction is calculated as amount of energy consumption in the vertical direction=(weight×gravity×distance in the vertical direction). Further, the amount of energy consumption in the running direction is calculated as amount of energy consumption in the running direction=[weight×{(maximum speed in the running direction after kicking)2−(minimum speed in the running direction after landing)2}/2]. Further, the amount of energy consumption in the horizontal direction is calculated by amount of energy consumption in the horizontal direction=[weight×{(maximum speed in the horizontal direction after kicking)2−(minimum speed in the horizontal direction after landing)2}/2].

### Landing Shock

Landing shock is an exercise index indicating how much shock is applied to a body due to landing. The landing shock is calculated by landing shock=(shock force in the vertical direction+shock force in the running direction+shock force in the horizontal direction). Here, the shock force in the vertical direction=(weight×speed in the vertical direction at the time of landing/shock time).

Further, the shock force in the running direction={weight×(speed in the running direction before landing−minimum speed in the running direction after landing)/shock time}. Further, shock force in the horizontal direction={weight×(speed in the horizontal direction before landing−minimum speed in the horizontal direction after landing)/shock time}.

### Running Capability

Running capability is an exercise index of running power of the user. For example, a ratio of the stride and the ground time is known to have a correlation with the running record (time) (“About Ground Time and time of separation from ground During 100 m Race”, Journal of Research and Development for Future Athletics. 3(1): 1-4, 2004.). The running capability is calculated using running capability=(stride/ground time).

### Anteversion Angle

An anteversion angle is an exercise index indicating how much the torso of the user is inclined with respect to the ground. The anteversion angle in a state in which the user stands perpendicular to the ground is 0, the anteversion angle when the user slouches is a positive value, and the anteversion angle when the user leans back is a negative value. The anteversion angle is obtained by converting the pitch angle of them frame to be the specification as described above. When the exercise analysis device 2 (inertial measurement unit 10) is mounted on the user, there is a possibility that there is already a slope, and thus, a time of rest is assumed to be a 0 degree, and the anteversion angle may be calculated using a resultant amount of change.

### Degree of Timing Matching

A degree of timing matching is an exercise index indicating how close the timing of the feature point of the user is to a good timing. For example, an exercise index indicating how close a timing of waist rotation is to a timing of kicking is considered. In a running way in which the leg is flowing, since one leg still remains behind the body when the other leg lands, the running way in which the leg is flowing can be determined when the rotation timing of the waist comes after the kicking. The waist rotation timing substantially matches the timing of the kicking, and the running way is said to be good. On the other hand, the waist rotation timing is later than the timing of the kicking, and the running way is said to be a way in which the leg is flowing.

### 3-7. Second Analysis Information

Hereinafter, each item of the second analysis information 354 calculated by the second analysis information generation unit 276 will be described in detail.

### Energy Loss

An energy loss is an exercise index indicating an amount of energy wasted in an amount of energy consumed by one-step advance, and also indicates integration in a running period of an amount of energy wasted in the amount of energy consumed by one-step advance. The energy loss is calculated by energy loss={amount of energy consumption×(100−directly-under landing rate)×(100−propulsion efficiency)}. Here, the directly-under landing rate is any one of directly-under landing rates 1 to 3, and the propulsion efficiency is any one of propulsion efficiencies 1 to 4.

### Energy Efficiency

Energy efficiency is an exercise index indicating whether the energy consumed by one-step advance is effectively used as energy for advance in the running direction, and also indicates integration in the running period. The energy efficiency is calculated as energy efficiency={(amount of energy consumption−energy consumption loss)/amount of energy consumption}.

### Load on Body

A load on the body is an exercise index indicating how much shock is applied to the body through accumulation landing shock. Since injury is caused due to the accumulation of the shock, ease of injury can be determined by evaluating the load on the body. The load on the body is calculated as the load on the body=(load on a right leg+load on a left leg). The load on the right leg can be calculated by integrating landing shock of the right leg. The load on the left leg can be calculated by integrating landing shock of the left leg. Here, for the integration, both integration during running and integration from the past can be performed.

### 3-8. Left-Right Difference Ratio (Left-Right Balance)

The left-right difference ratio 355 is an exercise index indicating how much the left and right of the body are different from each other for the running pitch, the stride, the ground time, the shock time, each item of the first analysis information 353, and each item of the second analysis information 354, and is assumed to indicate how much the left leg is different from the right leg. The left-right difference ratio 355 is calculated as left-right difference ratio 355=(numerical value of left leg/numerical value of right leg×100) (%), and the numerical value is each numerical value of the running pitch, the stride, the ground time, the shock time, the amount of brake, the propulsion power, the directly-under landing rate, the propulsion efficiency, the speed, the acceleration, the running distance, the anteversion angle, the rotation angle of the waist, the rotation angular speed of the waist, the amount of inclination to left and right, the running capability, the amount of energy consumption, the energy loss, the energy efficiency, the landing shock, and the load on the body. Further, the left-right difference ratio 355 also includes an average value or a dispersion of each numerical value.

### 3-9. Procedure of Process

FIG. 13 is a flowchart diagram illustrating an example of a procedure of an exercise analysis process (exercise analysis method) performed by the processing unit 20 of the exercise analysis device 2 during running of the user according to the present embodiment. The processing unit 20 executes the exercise analysis program 300 stored in the storage unit 30 to execute the exercise analysis process in the procedure of the flowchart of FIG. 13.

As illustrated in FIG. 13, the processing unit 20 waits until the processing unit 20 receives a measurement start command (N in S10). When the processing unit 20 receives the measurement start command (Y in S10), the processing unit 20 first calculates an initial posture, an initial position, and an initial bias using the sensing data measured by the inertial measurement unit 10 and the GPS data on the assumption that the user is at rest (S20).

Then, the processing unit 20 acquires the sensing data from the inertial measurement unit 10, and adds the acquired sensing data to the sensing data table 310 (S30).

Then, the processing unit 20 performs the inertial navigation operation process to generate operation data including various information (S40). An example of a procedure of this inertial navigation operation process will be described below.

Then, in an exercise information generation process in which exercise analysis information is generated when the user exercises, the processing unit 20 performs the exercise analysis information generation process using the calculation data generated in S40 and generates exercise analysis information (S50). An example of a procedure of this exercise analysis information generation process will be described below.

Then, the processing unit 20 generates the output information during running using the exercise analysis information generated in S50 and transmits the output information during running to the output unit 70 and the reporting device 3 (S60).

In an output process in which exercise analysis information is converted into predetermined perceptual information and the perceptual information is output, the output unit 70 converts the transmitted exercise analysis information (output information during running) into predetermined perceptual information and outputs the perceptual information (S65).

Also, the processing unit 20 repeats the process of S30 and subsequent steps each time the sampling period Δt elapses after the processing unit 20 acquires previous sensing data (Y in S70) until the processing unit 20 receives the measurement end command (N in S70 and N in S80).

When the processing unit 20 receives a running analysis start command (Y in S80), the processing unit 20 generates running result information using the exercise analysis information generated in S50, transmits the running result information to the reporting device 3 (S90), and ends the exercise analysis process.

FIG. 14 is a flowchart diagram illustrating an example of a procedure of the inertial navigation operation process (process of S40 in FIG. 13). The processing unit 20 (inertial navigation operation unit 22) executes the inertial navigation operation program 302 stored in the storage unit 30 to execute the inertial navigation operation process in the procedure of the flowchart of FIG. 14.

As illustrated in FIG. 14, first, the processing unit 20 removes the bias from the acceleration and the angular speed included in the sensing data acquired in S30 of FIG. 13 using the initial bias calculated in S20 in FIG. 13 (using the acceleration bias bₐ and the angular speed bias b_{ω} after the acceleration bias bₐ and the angular speed bias b_{ω} are estimated in S150 to be described below) to correct the acceleration and the angular speed, and updates the sensing data table 310 with the corrected acceleration and angular speed (S100).

The processing unit 20 then integrates the sensing data corrected in S100 to calculate a speed, a position, and a posture angle, and adds calculation data including the calculated speed, position, and posture angle to the operation data table 340 (S110).

The processing unit 20 then performs a running detection process (S120). An example of a procedure of this running detection process will be described below.

Then, when the processing unit 20 detects a running period through the running detection process (S120) (Y in S130), the processing unit 20 calculates a running pitch and a stride (S140). Further, when the processing unit 20 does not detect the running period (N in S130), the processing unit 20 does not perform the process of S140.

Then, the processing unit 20 performs an error estimation process to estimate the speed error δv^{e}, the posture angle error ε^{e}, the acceleration bias bₐ, the angular speed bias b_{ω}, and the position error δp^{e} (S150).

The processing unit 20 then corrects the speed, the position, and the posture angle using the speed error δv^{e}, the posture angle error ε^{e}, and position error δp^{e} estimated in S150, respectively, and updates the operation data table 340 with the corrected speed, position, and posture angle (S160). Further, the processing unit 20 integrates the speed corrected in S160 to calculate a distance of the e frame (S170).

The processing unit 20 then coordinate-transforms the sensing data (acceleration and angular speed of the b frame) stored in the sensing data table 310, the calculation data (the speed, the position, and the posture angle of the e frame) stored in the operation data table 340, and the distance of the e frame calculated in S170 into acceleration, angular speed, speed, position, posture angle, and distance of the m frame (S180).

Also, the processing unit 20 generates operation data including the acceleration, angular speed, speed, position, posture angle, and distance of the m frame after the coordinate transformation in S180, and the stride and the running pitch calculated in S140 (S190). The processing unit 20 performs the inertial navigation operation process (process of S100 to S190) each time the processing unit 20 acquires the sensing data in S30 of FIG. 13.

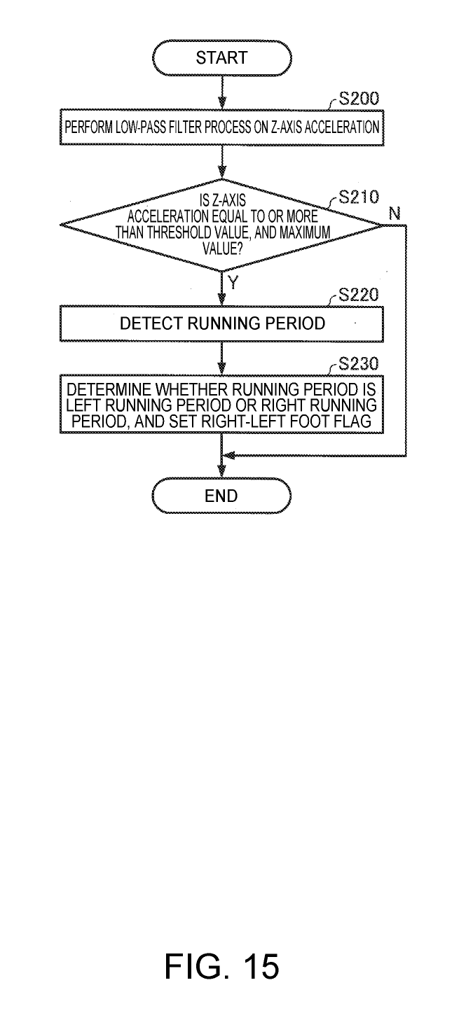

FIG. 15 is a flowchart diagram illustrating an example of a procedure of the running detection process (process of S120 in FIG. 14). The processing unit 20 (the running detection unit 242) executes the running detection process in the procedure of the flowchart of FIG. 15.

As illustrated in FIG. 15, the processing unit 20 performs a low-pass filter process on the z-axis acceleration included in the acceleration corrected in S100 of FIG. 14 (S200) to remove noise.

Then, when the z-axis acceleration subjected to the low-pass filter process in S200 is equal to or more than a threshold value and is a maximum value (Y in S210), the processing unit 20 detects a running period at this timing (S220).

Also, the processing unit 20 determines whether the running period detected in S220 is a left running period or a right running period, sets the right-left foot flag (S230), and ends the running detection process. When the z-axis acceleration is smaller than the threshold value or is not the maximum value (N in S210), the processing unit 20 ends the running detection process without performing the process of S220 and subsequent steps.

FIG. 16 is a flowchart diagram illustrating an example of a procedure of the exercise analysis information generation process (process of S50 in FIG. 13). The processing unit 20 (exercise analysis unit 24) executes the exercise analysis information generation program 304 stored in the storage unit 30 to execute the exercise analysis information generation process in the procedure of the flowchart of FIG. 16.

As illustrated in FIG. 16, first, the processing unit 20 calculates respective items of the basic information 352 using the operation data generated through the inertial navigation operation process in S40 of FIG. 13 (S300).

The processing unit 20 then performs a process of detecting the feature point (for example, landing, depression, or separation from ground) in the running exercise of the user using the operation data (S310).

When the processing unit 20 detects the feature point in the process of S320 (Y in S320), the processing unit 20 calculates the ground time and the shock time based on a timing of detection of the feature point (S330). Further, the processing unit 20 uses a part of the operation data and the ground time and the shock time generated in S330 as the input information 351, and calculates some items of the first analysis information 353 (item requiring information on the feature point for calculation) based on the timing of detection of the feature point (S340). When the processing unit 20 does not detect the feature point in the process of S310 (N in S320), the processing unit 20 does not perform the process of S330 and S340.

The processing unit 20 then calculates other items (items not requiring the information on the feature point for calculation) of the first analysis information 353 using the input information 351 (S350).

The processing unit 20 then calculates respective items of the second analysis information 354 using the first analysis information 353 (S360).

The processing unit 20 then calculates the left-right difference ratio 355 for each item of the input information 351, each item of the first analysis information 353, and each item of the second analysis information 354 (S370).

The processing unit 20 adds a current measurement time to respective information calculated in S300 to S370, stores the resultant information in the storage unit 30 (S380), and ends the exercise analysis information generation process.

### 4. Effect

The exercise analysis device 2 of the present embodiment includes the exercise analysis unit 24 that generates the exercise analysis information 350 during running or walking of the user using output of the inertial measurement unit (IMU) 10 and the output unit 70 that converts the periodically generated exercise analysis information 350 into perceptual information such as sound, light, or vibration and outputs the perceptual information in synchronization with landing. For this reason, the exercise analysis information 350 is converted and reported as perceptual information such as sound, light, or vibration, and thus it is possible for the user to easily check the exercise analysis information 350 during running or walking of the user. Accordingly, it is possible for the user, for example, to modify an exercise state during running or walking according to the exercise analysis information 350.

In addition, since the perceptual information is at least one of sound, light, or vibration, it is possible to check the exercise analysis information 350 sensately without relying on vision. Since the sound includes mimetic sound and the perceptual information becomes easier to hear, it is possible to more accurately check the exercise analysis information 350.

Further, the exercise analysis information 350 is converted into sound, light, or vibration having a frequency corresponding to values of the exercise analysis information 350 and is output, and thus it is possible to check a difference or magnitude of the values of the exercise analysis information 350 as a difference or magnitude of the frequency. In addition, since a relationship between the values of the exercise analysis information 350 and an output time interval an output magnitude of the perceptual information or the like is a comparison relationship or a reverse-comparison relationship, it is possible to check a difference or magnitude of the values of the exercise analysis information 350.

Further, since the exercise analysis information 350 includes information related to a speed, an acceleration, stride, a pitch, propulsion efficiency, an amount of brake at the time of landing, or ground time during running or walking of the user, it is possible to check the exercise analysis information 350 of the user in detail.

In addition, the exercise analysis information 350 is output as different perceptual information on the left foot and the right foot of the user, and thus it is possible for the user to easily determine and check whether the exercise analysis information 350 is the exercise analysis information 350 on the left foot or the right foot.

Further, the exercise analysis information 350 converted into perceptual information may be output at a timing when the left foot or the right foot of the user during running or walking is landed or within ±100 ms at the time of the timing of the landing, and thus it is possible to check the exercise analysis information 350 while accurately maintaining rhythm of the user during running.

In exercise analysis system 1 of the present embodiment, since the exercise analysis device 2 includes the output unit 70 that converts the exercise analysis information 350 periodically generated based on output of the inertial measurement unit (IMU) 10 into perceptual information, and outputs the perceptual information in synchronization with landing, by outputting the exercise analysis information 350 as the perceptual information, the exercise analysis information 350 is converted and reported as perceptual information such as sound, light, or vibration. For this reason, it is possible for the user to easily check the exercise analysis information 350 during running or walking. Accordingly, it is possible for the user, for example, to modify an exercise state during running or walking according to the exercise analysis information 350.

Since the exercise analysis method of the present embodiment includes the output step that converts the exercise analysis information 350 periodically generated based on output of the inertial measurement unit (IMU) 10 into perceptual information, and outputs the perceptual information in synchronization with landing, by outputting the exercise analysis information 350 as the perceptual information, the exercise analysis information 350 is converted and reported as perceptual information such as sound, light, or vibration. For this reason, it is possible for the user to easily check the exercise analysis information 350 during running or walking.

The entire disclosure of Japanese Patent Application No. 2016-208385 filed Oct. 25, 2016 is expressly incorporated by reference herein.

What is claimed is:

1. An exercise analysis device comprising:
an exercise information generation unit that generates exercise information during running or walking of a subject using output of an inertial sensor; and
an output unit that converts exercise information periodically generated among the exercise information into predetermined perceptual information and outputs the perceptual information in synchronization with landing.

2. The exercise analysis device according to claim 1,
wherein the perceptual information is at least one of sound, light, or vibration.

3. The exercise analysis device according to claim 1,
wherein the exercise information includes information related to speed or acceleration in exercise of the subject.

4. The exercise analysis device according to claim 3,
wherein the exercise information is converted into sound, light, or vibration having a frequency corresponding to the speed or the acceleration and is output.

5. The exercise analysis device according to claim 1,
wherein the exercise information includes information related to a stride, a pitch, propulsion efficiency, an amount of brake at the time of landing, or ground time during running of the subject.

6. The exercise analysis device according to claim 1,
wherein the perceptual information is output within ±100 ms at the time of landing.

7. The exercise analysis device according to claim 2,
wherein the sound includes mimetic sound.

8. The exercise analysis device according to claim 1,
wherein the exercise information is output as different perceptual information on a left foot or a right foot of the subject.

9. An exercise analysis system comprising:
an exercise analysis device that includes an exercise information generation unit that generates exercise information during running or walking of a subject using output of an inertial sensor, and an output unit that converts exercise information periodically generated among the exercise information into predetermined perceptual information and outputs the perceptual information at the time of landing; and
a reporting device that reports the exercise information.

10. An exercise analysis method comprising:
generating exercise information during running or walking of a subject using output of an inertial sensor; and
converting exercise information periodically generated among the exercise information into predetermined perceptual information and outputting the perceptual information at the time of landing.
